(19) United States
(12) Patent Application Publication
(10) Pub. No.: US 2024/0000960 A1
(43) Pub. Date: Jan. 4, 2024

(54) ANTI-CD6 ANTIBODY CONJUGATES FOR TREATING T-CELL MEDIATED DISORDERS AND T-CELL LYMPHOMA/LEUKEMIA

(71) Applicant: The Cleveland Clinic Foundation, Cleveland, OH (US)

(72) Inventor: Feng Lin, Cleveland, OH (US)

(21) Appl. No.: 18/252,928

(22) PCT Filed: Nov. 16, 2021
(86) PCT No.: PCT/US2021/059482

§ 371 Date: May 15, 2023

Related U.S. Application Data
(60) Provisional application No. 63/114,300, filed on Nov. 16, 2020.

(51) Int. Cl. A61K 47/68; A61P 37/02

(52) U.S. Cl. CPC A61K 47/68031; A61P 37/02; A61K 47/6849

(57) ABSTRACT

Provided herein are compositions, systems, kits, and methods for treating a subject having a T-cell mediated disorder, B1-cell mediated disorder, a T-cell lymphoma, or a B-cell lymphoma, with an antibody drug conjugate (ADC) composed of an anti-CD6 antibody (or CD6 binding portion thereof) and a mitotic inhibitor drug (e.g., monomethyl auristatin E (MMAE)). In certain embodiments, the ADC further comprises a cleavable linker (e.g., protease cleavable linker) connecting the antibody component to the mitotic inhibitor drug component. In some embodiments, the subject is a human with autoimmune uveitis or Mantle cell lymphoma.

The present application claims priority to U.S. Provisional application serial number 63/114,300 filed Nov. 16, 2020, which is herein incorporated by reference in its entirety.

### STATEMENT REGARDING FEDERAL FUNDING

This invention was made with government support under EY025373 and EY033243 awarded by National Institutes of Health. The government has certain rights in the invention.

### SEQUENCE LISTING

The text of the computer readable sequence listing filed herewith, titled “38968-601_SEQUENCE_LISTING_ST25”, created Nov. 16, 2021, having a file size of 22,322 bytes, is hereby incorporated by reference in its entirety.

### FIELD

Provided herein are compositions, systems, kits, and methods for treating a subject having a T-cell or B1-Cell mediated disorder, or T-cell or B1-cell neoplasia, with an antibody drug conjugate (ADC) composed of an anti-CD6 antibody (or CD6 binding portion thereof) and a mitotic inhibitor drug (e.g., monomethyl auristatin E (MMAE)). In certain embodiments, the ADC further comprises a cleavable linker (e.g., protease cleavable linker) or uncleavable linker, connecting the antibody component to the mitotic inhibitor drug component. In some embodiments, the subject is a human with autoimmune uveitis or GVHD or T cell lymphoma or B cell lymphoma.

### BACKGROUND

Pathogenic T cells cause many diseases including most autoimmune diseases, graft-versus-host disease (GVHD) and transplantation rejection. Selective targeting these pathogenic T cells while sparing the normal T cells and other tissues is the “holy grail” of therapeutics development in modern medicine. So far, pan-immunosuppressive drugs such as corticosteroids are used to treat these patients, with limited efficacies and severe adverse effects.

It is also well-established that these pathogenic T cells, being reactive for self-or allogeneic antigens, once activated, start to actively proliferate to cause tissue damage while the other normal T cells remain quiescent. Thus selectively eliminating the proliferating T cells while leaving the quiescent T cells alone would be an effective strategy to develop new targeted drugs for diseases mediated by the pathogenic T cells.

### SUMMARY

Provided herein are compositions, systems, kits, and methods for treating a subject having a T-cell mediated disorder, a B1-cell mediated disorder, a T-cell lymphoma, or a B-cell lymphoma, with an antibody drug conjugate (ADC) composed of an anti-CD6 antibody (or CD6 binding portion thereof) and a mitotic inhibitor drug (e.g., monomethyl auristatin E (MMAE)). In certain embodiments, the ADC further comprises a cleavable linker (e.g., protease cleavable linker) connecting the antibody component to the mitotic inhibitor drug component. In some embodiments, the subject is a human with autoimmune uveitis or Mantle cell lymphoma.

In some embodiments, provided herein are methods of treating a subject comprising: administering antibody drug conjugate (ADC) to a subject with a disorder, wherein said disorder is a T-Cell mediated disorder, a B1-cell mediated disorder, a T-cell lymphoma, or a B-cell lymphoma, and wherein said ADC comprises: a) an anti-CD6 antibody, or CD6 binding portion thereof, and b) a mitotic inhibitor drug.

In certain embodiments, provided herein are compositions comprising: an antibody drug conjugate (ADC) comprising: a) an anti-CD6 antibody, or CD6 binding portion thereof, and b) a mitotic inhibitor drug.

In particular embodiments, the mitotic inhibitor drug comprises monomethyl auristatin E (MMAE). In other embodiments, the mitotic inhibitor drug is selected from the group consisting of: vincristine, eribulin, paclitaxel, paclitaxel protein-bound, docetaxel, estramustine, etoposide, ixabepilone, cabazitaxel, vincristine liposome, vinorelbine, vincristine, paclitaxel, etoposide, vinblastine, etoposide, and teniposide.

In some embodiments, the T-cell mediated disorder comprises autoimmune uveitis. In other embodiments, the T-Cell mediated disorder is selected from the group consisting of: rheumatoid arthritis (RA), type 1 diabetes, Multiple sclerosis, Celiac disease, graft versus host disease and Sjögren's syndrome. In additional embodiments, the ADC further comprises a cleavable linker (e.g., protease cleavable linker). In other embodiments, the anti-CD6 antibody, or CD6 binding portion thereof, comprises one or more (e.g., 1, 2, 3, 4, 5, or 6) CDRs from Table 1 (e.g., from antibody 1, 2, 3, 4, 5, 6, 7, or 8). In certain embodiments, the anti-CD6 antibody, or CD6 binding portion thereof, comprises one or more variable regions shown in FIGS. 18-21 or 30. In other embodiments, the subject is a human.

In certain embodiments, the ADC is administered to said subject at a dosage of about 0.1-20 mg/kg (e.g., about 0.1, 0.5, 0.8, 1.0, 1.3, 1.5, 1.7, 5 . . . 10 . . . 15 or 20 mg per kg of subject). In further embodiments, the subject has said B1-cell lymphoma. In other embodiments, the B1-cell lymphoma is Mantle cell lymphoma. In additional embodiments, the subject has said T-cell lymphoma.

In some embodiments, provided herein are in vitro systems comprising: a) an antibody drug conjugate (ADC) comprising: i) an anti-CD6 antibody, or CD6 binding portion thereof, and ii) a mitotic inhibitor drug; and b) a T-cell lymphoma cell, or a B-cell (e.g., B1-cell) lymphoma cell. In particular embodiments, the cell is in a culture dish.

In further embodiments, employed herein is a system comprising: a) an antibody drug conjugate (ADC) comprising: i) an anti-CD6 antibody, or CD6 binding portion thereof, and ii) a mitotic inhibitor drug; and b) instructions for treating a subject with said ADC, wherein said subject has a T-cell mediated disorder, a B1-cell mediated disorder, a T-cell lymphoma, or a B-cell lymphoma.

### DESCRIPTION OF THE FIGURES

The patent or application file contains at least one drawing executed in color. Copies of this patent or patent application publication with color drawings will be provided by the Office upon request and payment of the necessary fee.

FIG. 1 shows a diagram of an ADC.

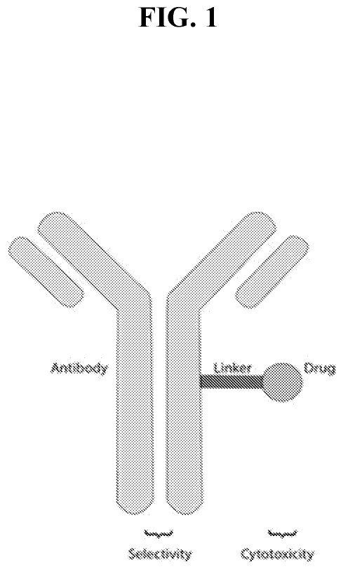

FIG. 2 shows CD6 is an established cell surface marker of T cells that binds to its ligands, CD166 and CD318.

FIG. 3 shows an identification and humanization of the high-affinity anti-CD6 mAb.

FIG. 4 shows the anti-CD6 mAb is efficiently internalized by T cells as measured by detecting the activated pHAmine fluorescence using a flow cytometer after 4 hours of incubation at 37° C.

FIG. 5 shows development of a CD6-targeted ADC. A. Diagram of the CD6-targeted ADC with conjugated MMAE through a cleavable linker. B. CD6-ADC potently kills proliferating T cells in vitro with an IC50 of 0.5 nM. A T cell line (HH cells) were incubated with different concentrations of CD6-ADC (ADC), or the anti-CD6 mAb (CD6) or the control IgG (IgG). Cell death was assessed at different time points. Representative results of 4 experiments.

FIG. 6: CD6-ADC kills proliferating T cells in vitro as measured by MTT assays. Different concentrations of CD6-ADC were incubated with HH cells, a T cell line in vitro. The viabilities of the T cells were quantitated at different time points by a MTT assay.

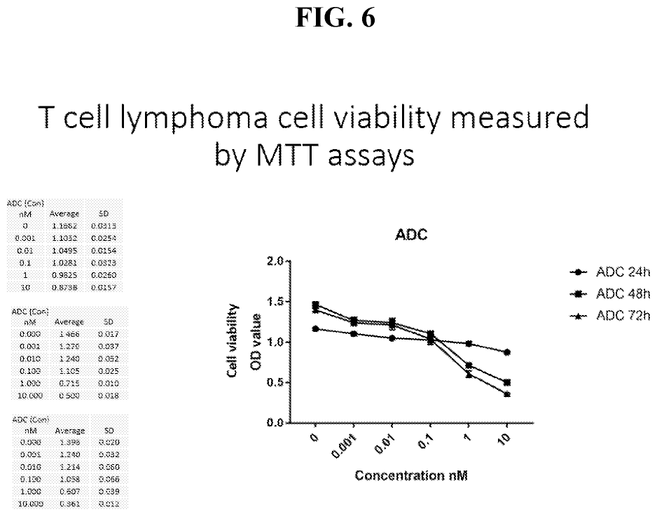

FIG. 7: CD6-ADC kills proliferating T cells in vitro as measured by a PI-incorporation assays Different concentrations of CD6-ADC were incubated with HH cells, a T cell line in vitro. The viabilities of the T cells were quantitated at different time points by a PI assay.

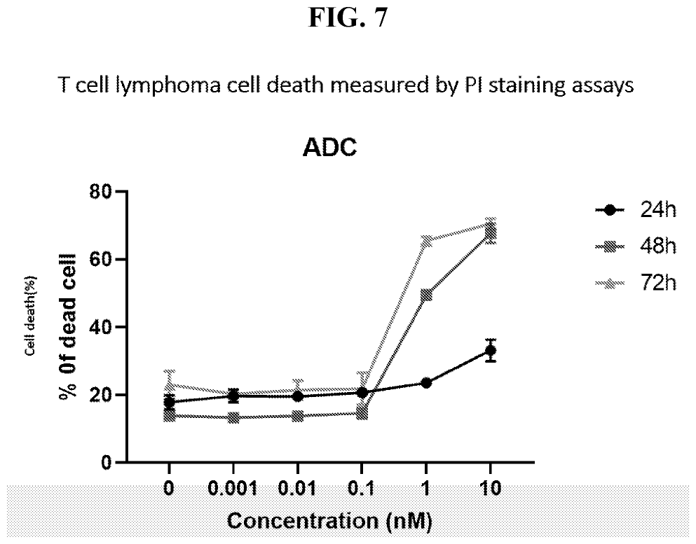

FIG. 8: CD6-ADC kills proliferating T cells in vitro as measured by MTT assays. IC50 at 72 hr was calculated to be˜0.4 nM.

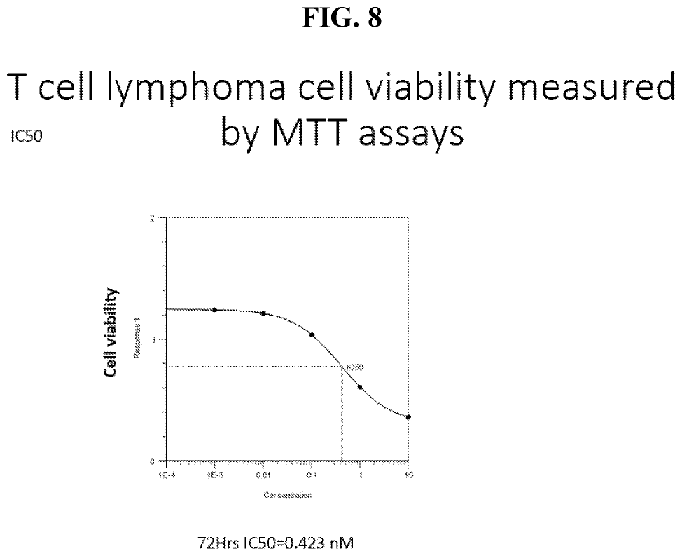

FIG. 9: CD6-ADC kills proliferating T cells in vitro as measured by trypan blue assays. Different concentrations of CD6-ADC (ADC), naked anti-CD6 mAb (CD6) and control IgG (IgG) were incubated with HH cells, a T cell line in vitro. The viabilities of the T cells were quantitated at different time points by a trypan blue assay.

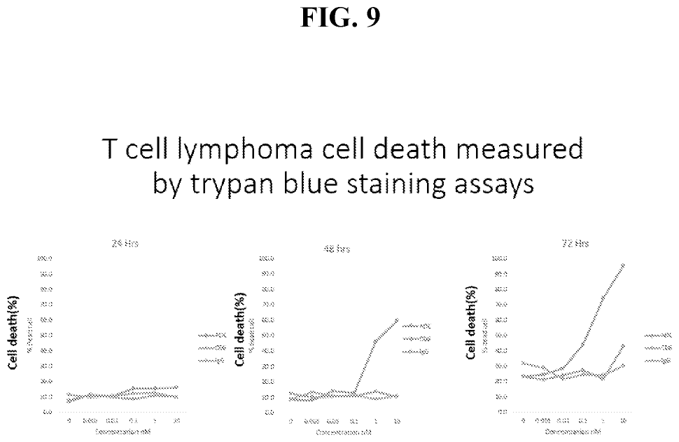

FIG. 10: The “naked” anti-CD6 mAb does not kill the proliferating T cells in vitro at low concentrations. Different concentrations of the anti-CD6 mAb (UMCD6) were incubated with HH cells, a T cell line in vitro. The viabilities of the T cells were quantitated at different time points by a PI assay.

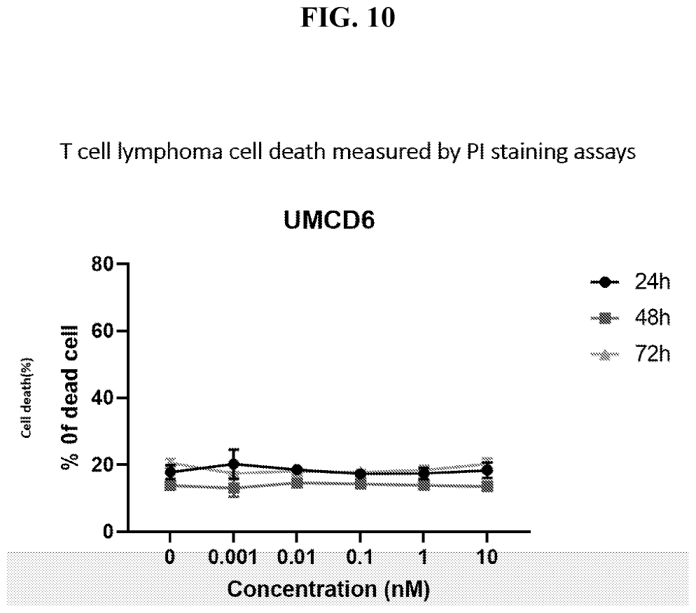

FIG. 11: The control non-specific IgGs do not kill the proliferating T cells in vitro at low concentrations. Different concentrations of the control IgG (IgG) were incubated with HH cells, a T cell line in vitro. The viabilities of the T cells were quantitated at different time points by a PI assay.

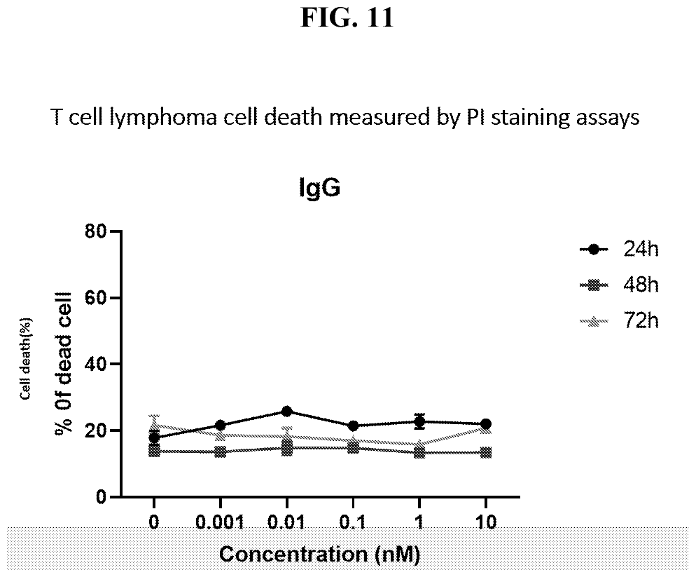

FIG. 12: WT mice were immunized with a retinal antigen IRBP to induce EAU (experimental autoimmune uveitis). Splenocytes were collected 10 days later and subjected to an antigen-specific T cell proliferation assay based on BrdU incorporation. All controls are shown here.

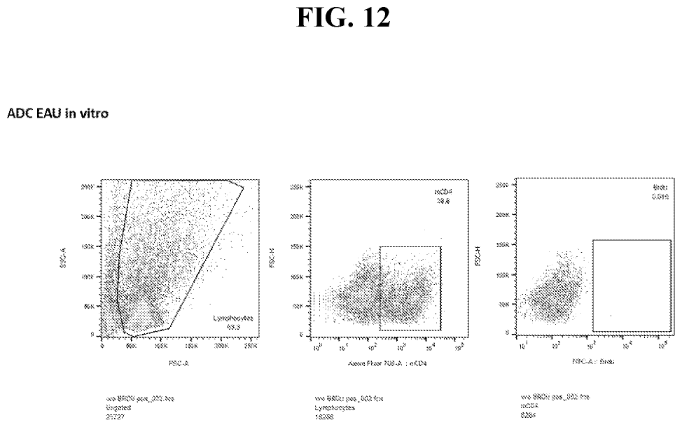

FIG. 13: The splenocytes from mouse #193 were cultured in the presence of different concentrations of the control IgG (IgG), naked anti-CD6 mAb (UMCD6) and the CD6-ADC (ADC). Antigen-specific proliferating T cells (BrdU+) were quantitated by flow, showing that the CD6-ADC but not the control IgG nor the UMCD6 eliminated the antigen-specific (uveitogenic T cells) in a concentration-dependent manner.

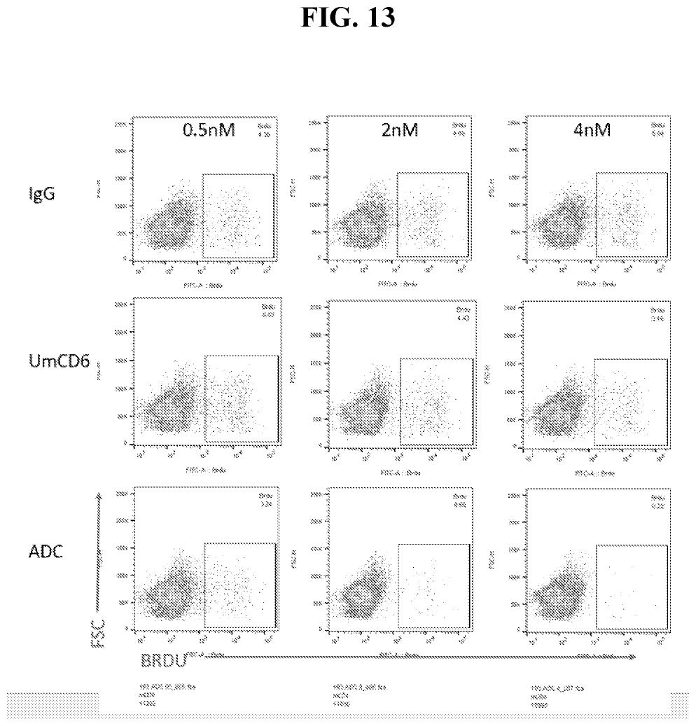

FIG. 14: The splenocytes from mouse #195 were cultured in the presence of different concentrations of the control IgG (IgG), naked anti-CD6 mAb (UMCD6) and the CD6-ADC (ADC). Antigen-specific proliferating T cells (BrdU+) were quantitated by flow, showing that the CD6-ADC but not the control IgG nor the UMCD6 eliminated the antigen-specific (uveitogenic T cells) in a concentration-dependent manner.

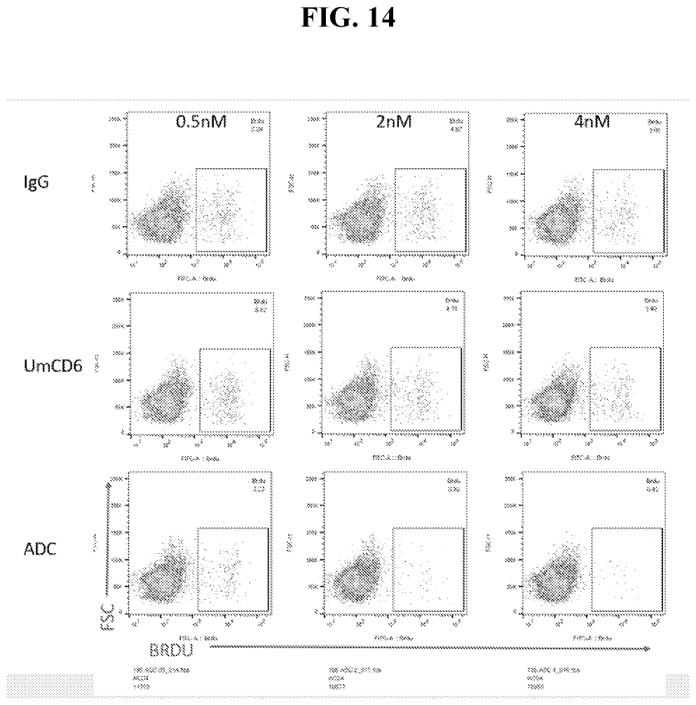

FIG. 15: The splenocytes from mouse #197 were cultured in the presence of different concentrations of the control IgG (IgG), naked anti-CD6 mAb (UMCD6) and the CD6-ADC (ADC). Antigen-specific proliferating T cells (BrdU+) were quantitated by flow, showing that the CD6-ADC but not the control IgG nor the UMCD6 eliminated the antigen-specific (uveitogenic T cells) in a concentration-dependent manner.

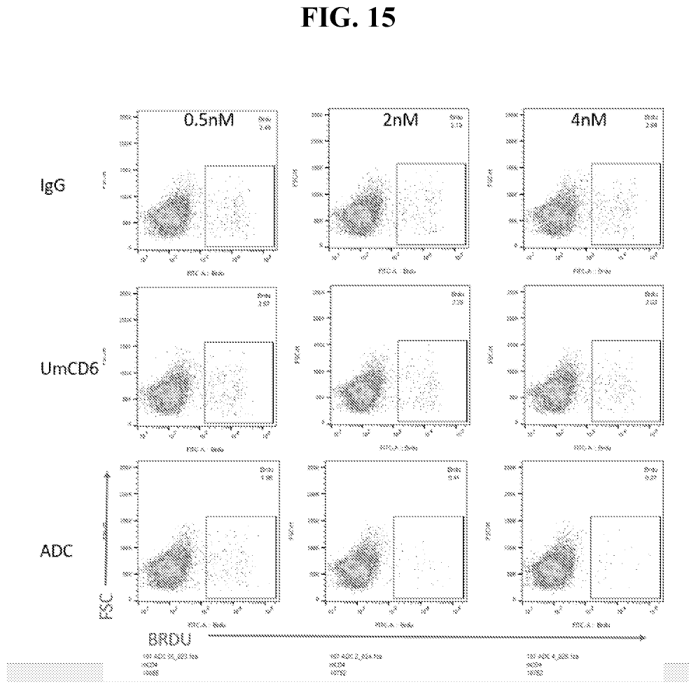

FIG. 16: Summary of the in vitro killing results.

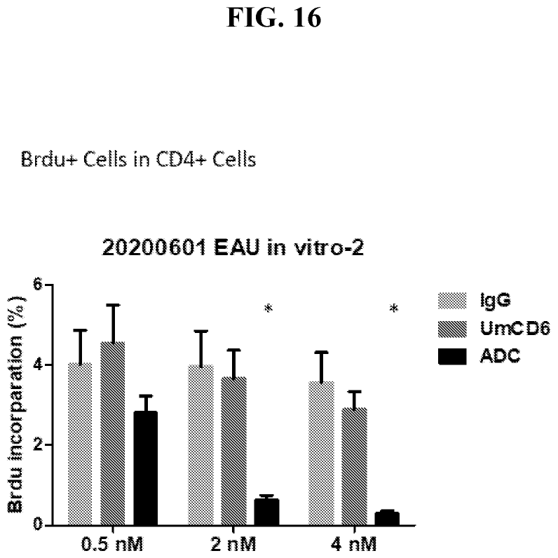

FIG. 17: CD6-ADC but not the naked anti-CD6 mAb nor the control IgG protects mice from EAU induced by the uvetiogenic T cells in vivo. In vitro expanded uveitogenic T Cells were adoptively transferred into naïve recipient mice per our established protocol. The recipient mice were then randomly divided into 3 groups and treated with 0.5 mg/kg of CD6-ADC (ADC), the naked anti-CD6 mAb (UMCD6) or the control IgG (IgG). The development and severity of the EAU were monitored daily by indirect ophthalmoscopy.

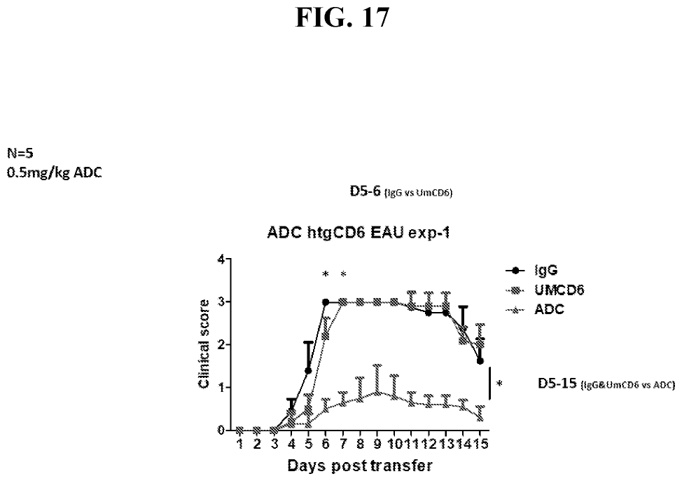

FIGS. 18A and 18B provide the (A) DNA and (B) amino acid sequences for the VH2-hIgG1CH antibody fragment (see, U.S. Pat. No. 10,562,975, herein incorporated by reference).

FIGS. 19A and 19B provide the (A) DNA and (B) amino acid sequences for the VH4-hIgG1CH antibody fragment (see, U.S. Pat. No. 10,562,975, herein incorporated by reference).

FIGS. 20A and 20B provide the (A) DNA and (B) amino acid sequences for the VH4-hIgG1CH antibody fragment (see, U.S. Pat. No. 10,562,975, herein incorporated by reference).

FIGS. 21A and 21B provide the (A) DNA and (B) amino acid sequences for the VL-hIgKCL antibody fragment (see, U.S. Pat. No. 10,562,975, herein incorporated by reference).

FIG. 22: CD6-ADC eliminates proliferating human T cells. A. CD6-ADC kills proliferating T cells but not B cells. HH cells (a T human cell line) and Raji cells (a human B cell line) were incubated with different concentrations of CD6-ADC or anti-CD6 IgG for 6 hours. Cells were washed with PBS and cultured for 48 or 72 hours and dead cells were detected by Trypan blue staining. B. CD6-ADC significantly decreases numbers of both human CD4 and CD8 T cells in a dose-dependent manner. B1. PBMCs from healthy donors were activated by anti-CD3 and anti-CD28 Abs for 5 days. Different concentrations (0.5, 2, 4 nM) of CD6-ADC, anti-CD6 IgG, and mIgG were added during the activation. The frequencies of CD4/CD8 positive cells were detected by flow cytometry. B2. BrdU was added to the culture media 16 hours before the cell collection on Day 5. Cells were stained with anti-BrdU Ab and the BrdU incorporation was analyzed by flow cytometry. B3. CFSE was used to label PBMCs for tracing cell proliferation and the CFSE dividing cells were detected by a flow cytometer. The numbers of each type of cell were calculated as followed: the total cell number in each well×frequencies of positive cells. C. Representative results of BrdU incorporation in CD4 and CD8 T cells with 4 nM CD6-ADC and controls.

FIG. 23. CD6-ADC kills activated antigen-specific T cells. Splenocytes from mice of aEAU model were re-stimulated with IRBP peptide in the presence of different concentrations (0.5, 2 ,4 nM) of CD6-ADC, anti-CD6 IgG and mIgG for 3 days. BrdU was added 16 hours before cell harvest. BrdU corporation was detected by flow cytometry. A. Representative results of BrdU incorporation CD4 positive cells. B. Summary results of 3 mice.

FIG. 24. 0.5 mg/kg CD6-ADC does not have significant effects on resting T cells in vivo. Naïve htgCD6 mice were injected with 0.5 mg/kg CD6-ADC intravenously. The frequencies of T cells in the peripheral blood were monitored by flow cytometry. A1. Percentages of CD3 T cells in lymphocytes. A2 and A3. Percentages of CD4 and CD8 T cells in CD3 T cells. N=3.

FIG. 25. Treatment of CD6-ADC alleviates experimental autoimmune uveitis induced by the adoptive transfer of uveitogenic T cells (tEAU). 0.5 mg/kg CD6-ADC or controls were given to htg CD6 tEAU mice on the same day of the induction. A and B. Mice with CD6-ADC treatment exhibited reduced clinical and histological scores. N=5 per group. C. Representative of images of topical endoscopic fundus imaging (TEFI), confocal scanning laser ophthalmoscope (cSLO) and spectral-domain optical coherence tomography (SD-OCT) in CD6-ADC-treated and control mice on Day 8 after transfer. CD6-ADC treated tEAU mice showed much less abnormality, including than control mice. D. Inflammation presented on fundus images was quantified. E. Representative histopathological images for CD6-ADC-treated and control mice with tEAU on Day 18. mIgG and anti-CD6 IgG-treated mice exhibited significant retinal folds and infiltrating cells in the vitreous, whereas the histopathological changes were mitigated in CD6-ADC-treated mice.

FIG. 26: Treatment of CD6-ADC reduces active experimental autoimmune uveitis (aEAU). htgCD6 mice were immunized with IRBP peptide to induce aEAU. A. On Day 6, images of confocal scanning laser ophthalmoscope (cSLO) showed infiltrated cells in the retina, which provided the rationale for staring treatments. B. Treatments of 0.5 mg/kg CD6-ADC or mIgG-ADC were administrated to mice with aEAU every three days from Day 6. Mice with CD6-ADC treatments exhibited reduced clinical scores compared to mice with IgG-ADC treatments. N=6 per group. C. Representative of images of confocal scanning laser ophthalmoscope (cSLO) and spectral-domain optical coherence tomography (SD-OCT) in CD6-ADC-treated and control mice on Day 14 after immunization. D. Image quantification. E1. CD6-ADC treated mice showed reduced histological scores. E2. Representative histopathological images for CD6-ADC-treated and control mice with tEAU on Day 20. aEAU was alleviated by CD6-ADC treatments with fewer retinal folds and cell infiltrations.

FIG. 27: Treatment of CD6-ADC reduces the severity of GVHD induced by human PBMCs. GVHD model was induced in NSG mice with the injection of human PBMCs. 0.5 mg/kg CD6-ADC or mIgG-ADC was given to GVHD mice every three days from Day 3. A. The frequencies (A1 and A2) and absolute numbers (A3 and A4) of human CD45 and CD3 positive cells were decreased in the peripheral blood of mice with CD6-ADC treatments. Small inserts in each figure showed increasing human CD45 and CD3 positive cells in the first 3 days after inoculation. N=5 per group. B. Representative flow results of human CD45 and CD3 positive cells on Day 27. C. CD6-ADC treated mice eventually gained body weights, whereas the mIgG-ADC treated mice had weight loss during the progress of GVHD. D. CD6-ADC treated mice had reduced human CD45 and CD3 positive cells in both spleen (D1) and bone marrow (D2) than controls on Day 27. E. CD6-ADC treated mice had lower levels of IFN-gamma in the plasma than control mice on Day 12.

FIG. 28 shows representative scanned images of the MCL tissue arrays, from Example 2, stained with the anti-CD6 mAb. A. a slide that is part of the tissue array. B. a MCL biopsy specimen with CD6 staining; C. the same specimen with higher magnifications.

FIG. 29A shows MCL cell line SP53 is CD6+; pink: stained with isotype controls; blue: stained with anti-CD6 IgG. FIG. 29B shows CD6-ADC potently kills MCL cells in vitro. SP53 MCL cells were incubated with different concentrations of CD6-ADC or the control IgG-ADC for 72 hr. Cell death was assessed by trypan blue staining.

FIG. 30A shows the nucleic acid sequence (SEQ ID NO:18) of the heavy chain of monoclonal antibody UMCD6, with the framework regions in red and three CDRs in blue. FIG. 30B shows the amino acid sequence (SEQ ID NO:19) of the heavy chain of monoclonal antibody UMCD6, with the framework regions in red and the three CDRs in blue. FIG. 30C shows the nucleic acid sequence (SEQ ID NO:20) of the light chain of monoclonal antibody UMCD6, with the framework regions in red and three CDRs in blue. FIG. 30D shows the amino acid sequence (SEQ ID NO:21) of the light chain of monoclonal antibody UMCD6, with the framework regions in red and the three CDRs in blue. In certain embodiments, the variable regions from UMCD6 are employed (e.g., in a human-mouse chimeric antibody) in the systems, compositions, and methods herein. In other embodiments, just the six CDRs (e.g., engrafted on a human framework) are employed in the systems, compositions, and methods herein.

### DETAILED DESCRIPTION

Provided herein are compositions, systems, kits, and methods for treating a subject having a T-cell mediated disorder, a B-cell mediated disorder, a T-cell lymphoma, or a B-cell lymphoma, with an antibody drug conjugate (ADC) composed of an anti-CD6 antibody (or CD6 binding portion thereof) and a mitotic inhibitor drug (e.g., monomethyl auristatin E (MMAE)). In certain embodiments, the ADC further comprises a cleavable linker (e.g., protease cleavable linker) connecting the antibody component to the mitotic inhibitor drug component. In some embodiments, the subject is a human with autoimmune uveitis or Mantle cell lymphoma.

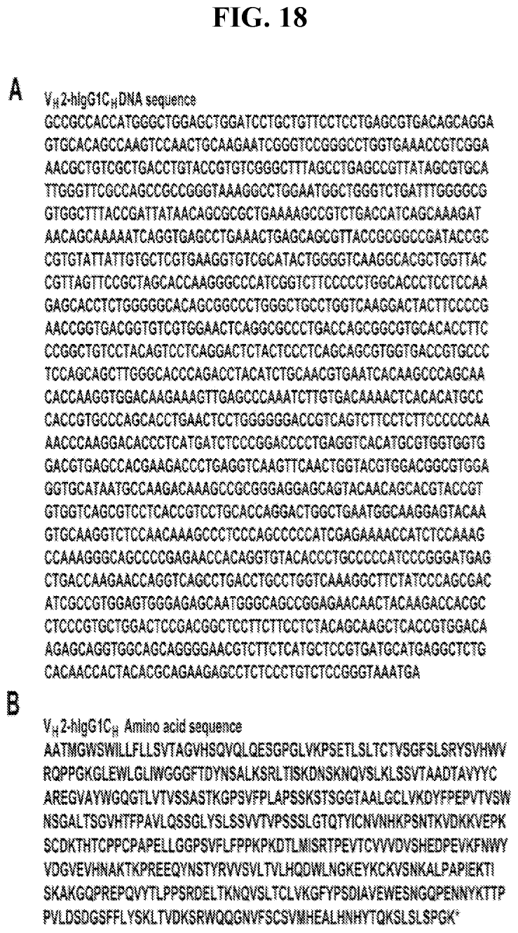

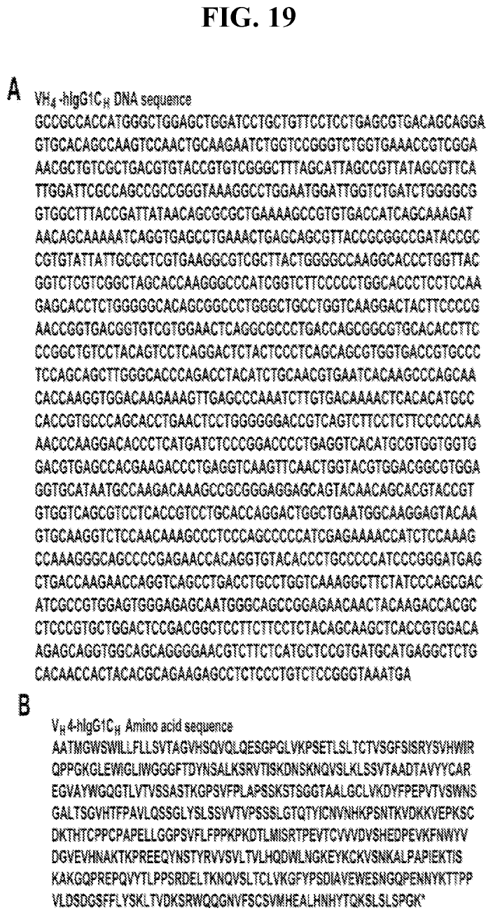

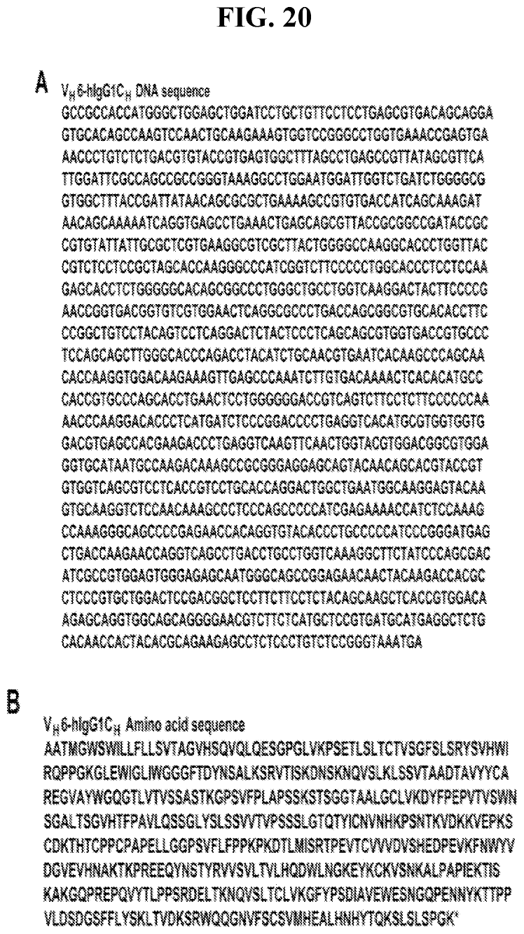

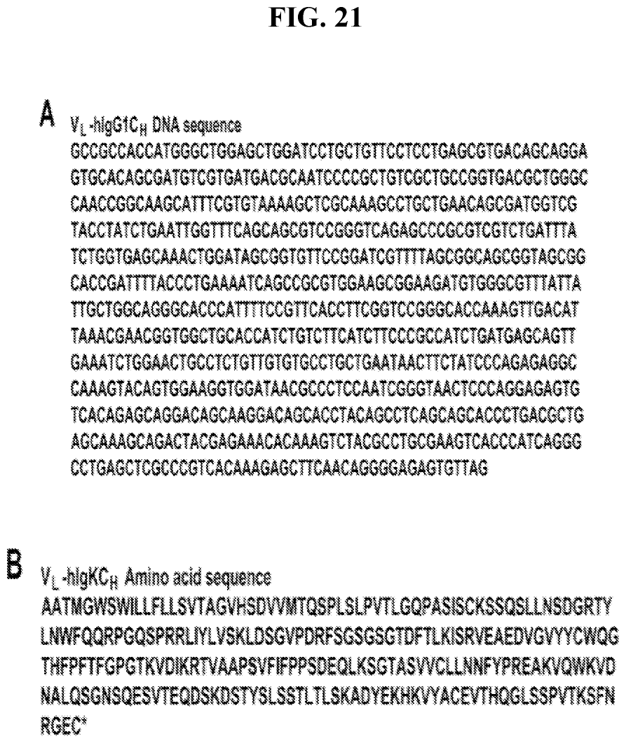

In work conducted during the development of embodiments described herein, we developed a T cell-targeted antibody drug conjugate (ADC) by conjugating a latent MMAE (monomethyl auristatin E), a clinically-proven anti-mitotic drug, onto a monoclonal antibody (mAb) against CD6 as described in U.S. Pat. No. 10,562,975, and in FIGS. 18-21.

In some embodiments, only the light and heavy chain variable regions, or just the CDRs, from the antibodies described in U.S. Pat. No. 10,562,975, and in FIGS. 18-21, are employed. In certain embodiments, the ADCs herein employs other anti-CD6 antibodies and antigen binding portions thereof, such as those known in the art (e.g., Itolizumab or LS-B9829 from LS Bio; UMCD6 or chimeric version thereof, see Singer, et al., Immunology 88(4): 537-543 (1996), herein incorporated by reference in its entirety). An internet search of PubMed and the USPTO patent literature can be employed to find other anti-CD6 antibodies and fragments thereof, particularly human or humanized antibodies). In other embodiments, one, two, three, four, five, or six CDRs (underlined) from any of the eight VH or eight VL chains from U.S. Pat. No. 10,562,975 are employed, as shown in Table 1 below. The humanized antibodies are numbered 1-8 in Table 1 below, each with a heavy chain and a light chain. In certain embodiments, the ADCs herein use the collection of 6 CDRs (underlined) from antibody 1, 2, 3, 4, 5, 6, 7, or 8.

TABLE 1

| Name | | Sequence |
|---|---|---|
| 1 | VH | QVQLQESGPGLVKPSETLSLTCTVSGGSISRYSVHWIRQPPGKGLEWIGLIWGGGFTD |
| | | YNSALKSRVTISVDTSKNQFSLKLSSVTAADTAVYYCAREGVAYWGQGTLVTVSS (SEQ ID NO: 1) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 2) |
| 3 | VH | QVQLQESGPGLVKPSETLSLTCTVSGGSISRYSVHWIRQPPGKGLEWIGLIWGGGFTD |
| | | YNSALKSRVSITVDTSKNQFSLKLSSVTAADTAVYYCAREGVAYWGQGTLVTVSS (SEQ ID NO: 3) |
| | VL | DVVMTQSPLSLPVTLGQPASISCKSSQSLLNSDGRTYLNWFQQRPGQSPKRLIYLVSK |
| | | LDSGVPDRFSGSGGTDFTLKISRVEAEDVGVYYCWQGTHFPFTFGPGTKVDIK (SEQ ID NO: 4) |
| 2 | VH | QVQLQESGPGLVKPSETLSLTCTVSGFSLSRYSVHWVRQPPGKGLEWLGLIWGGGF |
| | | TDYNSALKSRLTISKDNSKNQVSLKLSSVTAADTAVYYCAREGVAYWGQGTLVTV |
| | | SS |
| | | (SEQ ID NO: 5) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 6) |
| 4 | VH | QVQLQESGPGLVKPSETLSLTCTVSGFSISRYSVHWIRQPPGKGLEWIGLIWGGGFTD |
| | | YNSALKSRVTISKDNSKNQVSLKLSSVTAADTAVYYCAREGVAYWGQGTLVTVSS (SEQ ID NO: 7) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 8) |
| 5 | VH | QVQLQESGPGLVKPSETLSLTCTVSGGSLSRYSVHWVRQPPGKGLEWLGLIWGGGF |
| | | TDYNSALKSRLTISVDTSKNQFSLKLSSVTAADTAVYYCAREGVAYWGQGTLVTVS |
| | | S |
| | | (SEQ ID NO: 9) |
| | VL | DVVMTQSPLSLPVTLGQPASISCKSSQSLLNSDGRTYLNWFQQRPGQSPRRLIYLVSK |
| | | LDSGVPDRESGSGGTDFTLKISRVEAEDVGVYYCWQGTHFPFTFGPGTKVDIK (SEQ ID NO: 10) |
| 6 | VH | QVQLQESGPGLVKPSETLSLTCTVSGFSLSRYSVHWIRQPPGKGLEWIGLIWGGGFT |
| | | DYNSALKSRVTISKDNSKNQVSLKLSSVTAADTAVYYCAREGVAYWGQGTLVTVS |
| | | S |
| | | (SEQ ID NO: 11) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 12) |
| 7 | VH | QVQLQESGPGLVKPSETLSLTCTVSGGSISRYSVHWVRQPPGKGLEWLGLIWGGGFT |
| | | DYNSALKSRLTISVDTSKNQFSLKLSSVTAADTAVYYCAREGVAYWGQGTLVTVSS (SEQ ID NO: 13) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 14) |
| 8 | VH | QVQLQESGPGLVKPSETLSLTCTVSGGSLSRYSVHWVRQPPGKGLEWIGLIWGGGFT |
| | | DYNSALKSRLTISVDTSKNQVSLKLSSVTAADTAVYYCAREGVAYWGQGTLVTVSS (SEQ ID NO: 15) |
| | VL | DVVMTQSPLSLPVTLGQPASISCKSSQSLLNSDGRTYLNWFQQRPGQSPRRLIYLVSK |
| | | LDSGVPDRFSGSGGTDFTLKISRVEAEDVGVYYCWQGTHFPFTFGPGTKVDIK (SEQ ID NO: 16) |

In certain embodiments, the antibody is a monoclonal antibody, or antigen binding fragment thereof, such as a Fab, F(ab)2, or scFv. In certain embodiments, the ADCs herein selectively deliver the conjugated MMAE into the T cells (e.g., when delivered to the eye of a human or delivery to a tumor or systemically) which are positive for CD6, and because only the autoreactive T cells are proliferating and the normal T cells are quiescent, the activated MMAE will selectively kill the autoreactive T cells from within, while leaves the normal T cells and other non-T cells unaffected. In some embodiments, various ADCs can be tested for selectivity and efficacy in ablating disease T-cells (e.g., uveitogenic T cells) and thereby treating a T-cell mediated disorder (e.g., autoimmune uveitis using experimental autoimmune uveitis (EAU) as a model in CD6 humanized mice).

In certain embodiments, the ADCs described herein selectively target the autoreactive T cells (e.g., in the uvea of the eye) while generally sparing the normal T cells and other cells. In some embodiments, the ADCs described herein are administered to a subject to treat any T-Cell mediated disorder, as well as T-Cell lymphoma. In certain embodiments, the ADCs herein provide an anti-CD6 mAb (or antigen binding fragment thereof) to selectively deliver the anti-mitotic MMAE drug payload into the T cells, and the conjugated anti-mitotic drug, MMAE, only generally kills actively proliferating cells. By combining these two selective approaches, only the pathogenic proliferating T cells are ablated while the quiescent normal T cells and other proliferating non-T cells are left unaffected or generally unaffected.

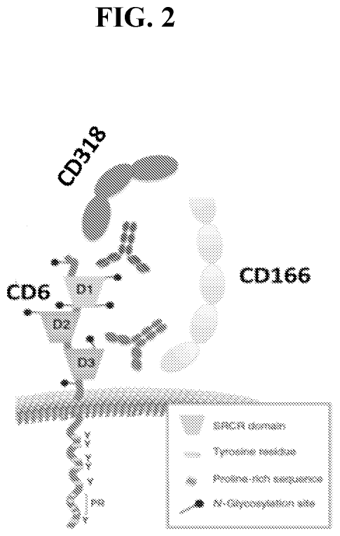

CD6, a protein containing 3 extracellular scavenger receptor cysteine-rich (SRCR) domains, (FIG. 2), was discovered over 30 years ago as a marker of T cells and has been suggested as a target for treating T cell-mediated autoimmune diseases, including multiple sclerosis (MS), rheumatoid arthritis, and Sjögren's syndrome. Recent interest in this field increased significantly when several groups discovered that CD6 is a risk gene for MS16-18, and itolizumab, an anti-CD6 mAb developed in Cuba, has been approved for treating psoriasis and COVID-19 in India (19,20). During the last 10 years, by developing and studying CD6 knockout (KO) mice, we have found that the lack of CD6 activity protected mice in several T cell-mediated autoimmune disease models, including models of autoimmune uveitis, MS and RA. These data strongly argue that CD6 is a key regulator of pathogenic T cell responses, and thus a potential therapeutic target. Indeed, we have identified, humanized and patented an anti-human CD6 mAb (U.S. Pat. No. 10,562,975) that is effective in treating these models of T cell-mediated diseases by directly suppressing T cell responses. As described below, we demonstrated that this humanized mAb binds to CD6 at a very high affinity (in the picomolar range), which is important for a successful ADC. Besides, we found that after binding to CD6 on T cells, this mAb can be quickly internalized, which is another key character for successful ADCs.

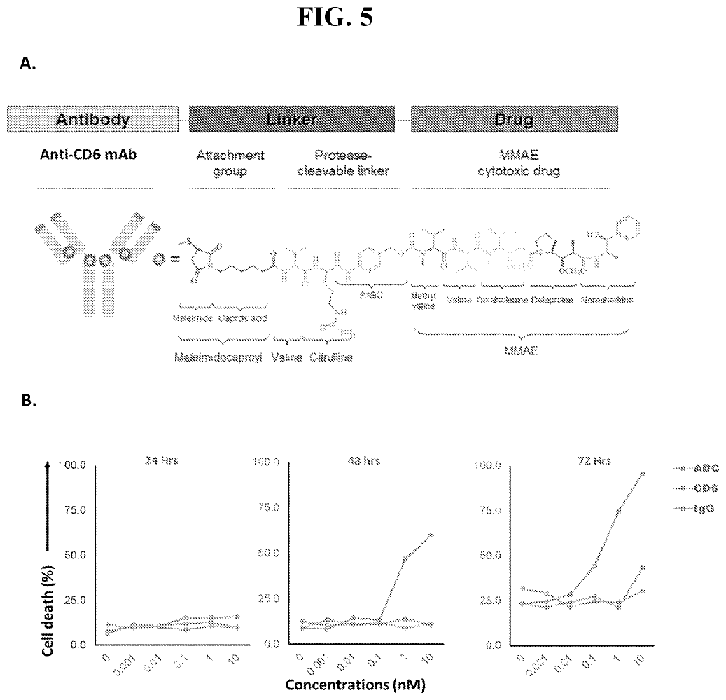

In work conducted during the development of embodiments herein, we generated an ADC by conjugating an inactivated form of the anti-mitotic drug, MMAE, onto our identified anti-CD6 mAb via a cleavable VC-PAB linker (FIG. 5). This ADC, by design, should selectively kill proliferating autoreactive T cells while sparing the normal T cells and other tissue cells. We determined that this novel ADC efficiently killed actively proliferating T cells in vitro (FIG. 5), demonstrating its potential as a new effective drug for autoimmune uveitis.

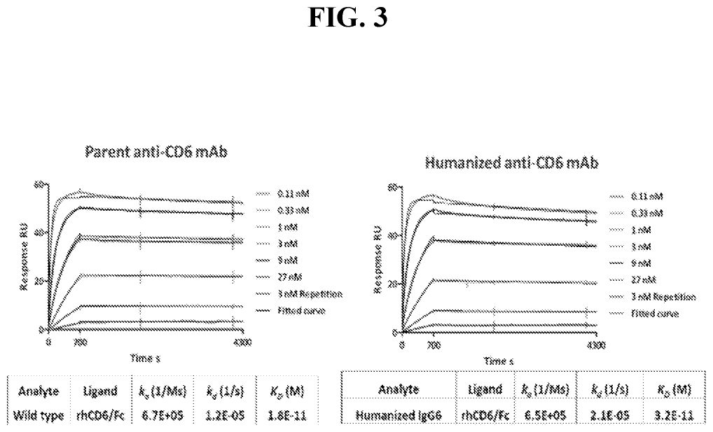

Humanization and characterization of the anti-human CD6 mAb. As part of the clinical development process, we humanized our identified mouse anti-human CD6 mAb via conventional complementarity determining region (CDR)-grafting technology, and compared the affinities of the mAb before and after humanization by surface plasmon resonance. As shown in FIG. 3, both the parent and the humanized anti-CD6 mAb have a very high affinity against CD6 with a KD of 10-11 M. In comparison, the affinity of the other anti-CD6 mAb, itolizumab, is reported to be 10-8 M.

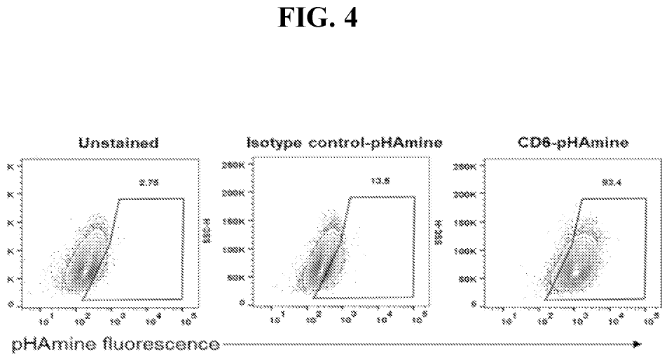

The anti-CD6 mAb is efficiently internalized by T cells. In addition to having a high affinity, another important feature for a mAb to be used for T cell-targeted ADC is its capacity to be internalized by T cells. We then first labeled the mAb with a pH-sensitive dye, pHAmine (Promega), which only becomes fluorescent after activation within intracellular acidic compartments and incubated it with a human T cell line, HH, followed by flow cytometric analysis. As shown in FIG. 4, we found that most of the T cells incubated with the pHAmine-labeled anti-CD6 mAb became fluorescent after incubation, demonstrating that the anti-CD6 mAb was efficiently internalized after binding to CD6 on the surface of the T cells.

In work conducted during development of embodiments herein, we developed a CD6-targeted ADC by conjugating the anti-mitotic drug, MMAE, onto the identified anti-CD6 mAb (FIG. 5). The target drug to antibody ratio is estimated to be 4 according to the spectroscopy analysis measuring OD418/OD280. To test this novel ADC in killing proliferating T cells, we incubated the HH T cells with different concentrations of the ADC, or the parent anti-CD6 mAb or the control IgG, and assessed cell death in 24, 48 and 72 hours by trypan blue staining. We found that the CD6-targeted ADC, but not the anti-CD6 mAb nor the control IgG, potently killed proliferating T cells with an IC50 of 0.5 nM in these in vitro assays (FIG. 5), indicating that it could be used for killing dividing autoreactive T cells in vivo.

### EXAMPLES

### Example 1

### CD6-Targeted Antibody-Drug Conjugate as Therapy for T Cell-Mediated Disorders

The selective targeting of pathogenic T cells is a “holy grail” in the development of new therapeutics for T cell-mediated disorders including many autoimmune diseases and graft-versus-host disease. In this Example, we describe the development of an exemplary CD6-targeted antibody-drug conjugate (CD6-ADC) by conjugating an inactive form of monomethyl auristatin E (MMAE), a potent mitotic toxin, onto a monoclonal antibody (mAb) against CD6, an established T cell surface marker. Even though CD6 is present on all T cells, the CD6-ADC is designed to selectively kill the pathogenic T cells that are actively dividing and thus susceptible to the anti-mitotic MMAE-mediated killing. We found that the CD6-ADC indeed selectively killed activated proliferating T cells while sparing the normal T cells both in human and in mouse. Furthermore, the same dose of CD6-ADC, but not the naked parent anti-CD6 mAb nor an IgG control nor a non-binding control IgG-ADC, efficiently treated two pre-clinical models of autoimmune uveitis and a model of graft-versus-host disease. These results provide solid evidence that the CD6-ADC could be used a pharmaceutical agent for the selective elimination of pathogenic T cells and thus a treatment of many T cell-mediated disorders.

### Methods and Materials

Generation of the CD6-ADC and control ADC: MMAE was conjugated onto the purified mouse anti-human CD6 IgG (UMCD6) and control mouse IgG via the VC-PAB linker using a kit (CellMosaic Inc, Boston, MA) followed by the manufacturer provided protocol. The target drug to antibody ratio of the resultant products was estimated by measuring OD418/OD280.

Human primary T cell killing assay: Human T cell killing assays were performed using human peripheral blood mononuclear cells (PBMCs). Unlabeled or Carboxyfluorescein succinimidyl ester (CFSE)-labeled PBMCs were seeded in the U-bottomed 96-well plate at a final concentration of 5×105 cells/ml in RPMI 1640 media (FBS 10%, Pen/Strep 100 μ/ml, L-glutamine 2 mM, HEPE 25 mM, sodium pyruvate 1 mM, (β-mercaptoethanol 50 μM, hIL-2 100 U/ml). T cells were either activated or activated with Dynabeads coupled with anti-CD3 and anti-CD28 antibodies (Abs) (ThermoFisher Scientific, USA) at a bead-to-cell ratio of 1:1, then incubated with 0.5, 2, and 4 nM of CD6-ADC, parental mouse anti-CD6 IgG or mouse IgG respectively for 5 days. For unlabeled PBMCs, 10 μM of bromodeoxyuridine (BrdU) was added to the culture media 16 hours before harvesting cells. The numbers of PBMCs were counted under the microscope and the frequencies of CD4 and CD8 T cells were detected by anti-mouse CD4 and anti-mouse CD8 mAbs (Biolegend, USA) by flow cytometry. To assess the T cell proliferation, BrdU incorporation (for unlabeled PBMCs) and CFSE dilution (for CFSE-labeled PBMCs) were analyzed using flow cytometry.

Human T cell line MOLT-4 killing assays: Human T cell line MOLT-4 (ATCC) which is actively proliferating under normal culture conditions were seeded at 40,000 cells/well in a 96-well plate in complete RPMI media containing 0, 0.1, 0.5, 2.5 or 12.5 nM of CD6-ADC or control ADC. After 6 hours of incubation, cells were washed and cultured in normal complete RPMI media for another 72 hours, then live and dead cells in each well were counted using a Countess Automatic Cell Counter (Invitrogen) after Trypan blue staining.

Antigen-specific T cell killing assay: Each of the CD6 humanized mice (8 to 12-week old) was subcutaneously immunized with a 200 ul complete Freund's adjuvant (CFA; Difco Laboratories, Inc., USA) containing 200 μg of the uveiogenic IRBP161-180 peptide (SGIPYIISYLHPGNTILHVD, SEQ ID NO:17; custom synthesized by GenScript USA Inc., USA) and 250 μg Mycobacterium tuberculosis H37Ra (Difco Laboratories, Inc., USA). Splenocytes from the immunized CD6 humanized mice were isolated 12 days later. 4×105 splenocytes were then re-stimulated with 20 μg/ml IRBP161-180 peptide, in the presence of 0.5, 2, and 4 nM of CD6-ADC, anti-CD6 IgG or mouse IgG respectively in RPMI 1640 media (FBS 10%, Pen/Strep 100 μ/ml, L-glutamine 2 mM) for 3 days. BrdU was added to the culture media 16 hours before collecting cells. Cells were stained with anti-mouse CD4 and anti-BrdU mAb (Biolegend), followed by analyses of BrdU incorporation in the CD4+ T cells using a flow cytometer.

CD6-ADC treatments of active and passive models of EAU: The inductions of active and passive models of EAU were performed as previously described in the literature. For the treatment of active EAU, immunized mice were treated by intraperitoneal injection of 0.5 mg/kg of CD6-ADC, anti-CD6 IgG or control IgG 6 day after immunization when clinical signs of uveitis developed; for the treatment of passive EAU, the recipient mice were treated the same way after adoptive transfer of the same numbers of pre-activated uveitogenic T cells. The development and severities of EAU were monitored daily using an indirect ophthalmoscope and assigned clinical scores of 0-4 according to previously published criteria (Caspi, R. R. (2003) Experimental autoimmune uveoretinitis in the rat and mouse. Curr. Protoc. Immunol. Chapter 15, Unit 15.6.).

Ocular imaging and histopathological analyses: Ocular imaging was performed as previously described (Zhang et al., J Leukoc Biol. 2016 Mar ;99(3):447-54; and Zhang et al., J Autoimmun . 2018 Jun;90:84-93.). In brief, under anesthesia and pupil dilation, mice were imaged by SD-OCT (Bioptigen, Inc., USA) and cSLO (HRA2/Spectralis, Heidelberg Engineering, Germany). SD-OCT imaging was performed with a 50 o field of view (FOV) to obtain cross-sectional images of the retina. cSLO images with a 55 o FOV were obtained with the optic nerve centrally positioned. cSLO was performed to measure the infrared (IR) reflectance and autofluorescence (AF) at the retina and outer retinal locations such as retinal pigmented epithelium. At the end of EAU studies, whole eyes were collected, fixed in 10% formalin solution for 48 h, and embedded in paraffin. 5 μm sections were cut through the pupil and optic nerve axis and stained with hematoxylin and eosin (H&E). The sections were assigned histopathological scores of 0-4 according to previously published criteria based on the inflammatory infiltration of and structural damage to the retina (Caspi, 2003).

CD6-ADC treatment of a model of GVHD: NSG mice (The Jackson Laboratory, USA, 8 weeks) were irradiated (200 rad) and given 3×10⁶ human PBMCs intravenously by tail vein injection to induce GVHD. Peripheral blood was collected every 3 days after the induction. Cells were stained with anti-mouse CD45, anti-human CD45, and anti-human CD3 mAbs and followed by flow cytometry analyses. Treatments of 0.5 mg/kg CD6-ADC and mIgG-ADC were administrated intraperitoneally every 3 days starting from D3 when increased numbers of human PBMCs was found in the peripheral blood indicating the start of a GVHD development. After 27 days, splenocytes and cells from bone marrow were isolated and the percentages of hCD45 and hCD3 positive cells in total white blood cells (mCD45 and hCD45 positive cells) were detected by a flow cytometer. The skin, spleen, liver, intestine, and colon were harvested, fixed in 10% formalin solution, embedded in paraffin, and stained with H&E.

### Results

### Development of a CD6-ADC and Non-Binding Control ADC Using an MMAE as the Payload

We generated the ADCs by conjugating the MMAE to the purified anti-CD6 IgG or mouse IgG via the VC-PAB linker using a commercially available kit following the manufacturer provided protocol. The target payload to antibody ratio is estimated to be approximately 3:1 according to the spectroscopy analysis measuring OD418/OD280. The prepared CD6-ADC and control ADC were aliquoted, lyophilized and stored in a −80° C. freezer until experiments.

### CD6- ADC Kills Activated Proliferating Human T Cells In Vitro

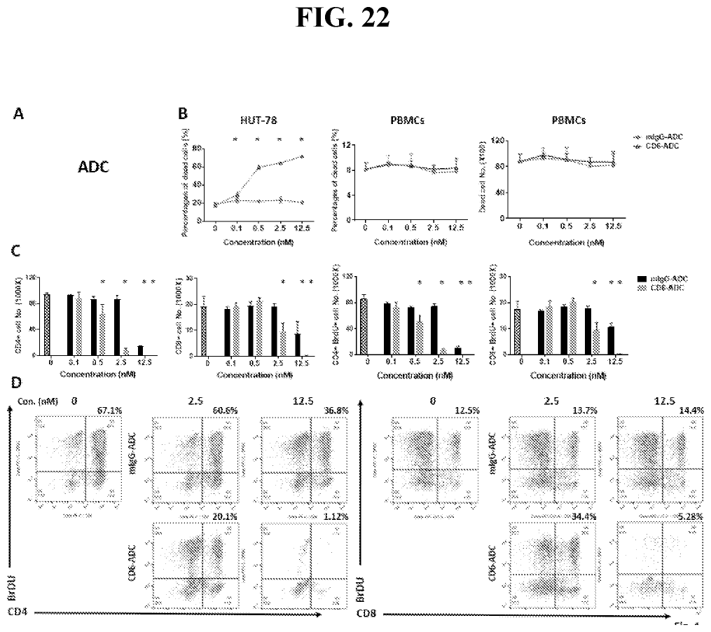

To demonstrate that CD6-ADC kills activated human T cells, we set up T cell killing assays using normal PBMC from healthy donors and activated the T cells within using Dyneabeads conjugated with anti-CD3 and anti-CD28 mAbs. We then incubated these cells with 0.5, 2, and 4 nM of the CD6-ADC, naked parental anti-CD6 IgG or control IgG respectively. On day 5, we quantitated the percentages and absolute numbers of proliferating CD4+ and CD8+ T cells in each well by flow cytometry using incorporated BrdU as a marker to identify the proliferating human T cells. See, FIG. 22. These studies showed that while the control ADC, the parental anti-CD6 IgG, or the control IgG had no measureable impact on the percentages and numbers of the proliferating CD4+ or CD8+ human T cells in all concentrations tested, CD6-ADC markedly reduced both the percentages and absolute numbers of these proliferating (BrdU+) human T cells within in a concentration-dependent manner even at the concentration of 0.5 nM (FIG. 22c).

### CD6- ADC Does Not Kill Normal Human T Cells in Vitro

To demonstrate that our CD6-ADC spares normal T cells which are quiescent, we directly incubated PBMCs from a healthy donor with 0-12.5 nM of CD6-ADC or control IgG-ADC, then measured T cell killing by flow cytometry using the LIVE/DEAD dye (Thermal Fisher) after gating on T cells (CD3+). See, FIG. 22. These studies showed that compared with the controls (green bars, T cells only), neither the control ADC nor the CD6-ADC had any significant detrimental effect on these normal primary human T cells even at the highest concentration tested (12.5 nM). Furthermore, there was no difference regarding the resultant dead T cell percentages or absolute numbers between the samples treated with control ADC (gray bars) or CD6-ADC (black bars) at all the concentrations tested. These studies provided direct evidence showing that our CD6-ADC does not kill normal human T cells.

### CD6-ADC Kills Proliferating T Cells but Spares Proliferating Non-T Cells in Vitro

To demonstrate that the CD6-ADC kills proliferating T cells but not other proliferating cells that do not express CD6, we again set up a cell-killing assay using a human T cell line MOLT-4 and a human B cell line Raji, both of which are actively dividing under normal culture conditions but only the T cell line expresses CD6 but not the Raji. We incubated the cells with 0, 0.1, 0.5, 2.5 or 12.5 nM of CD6-ADC, then assessed the cell killing by counting dead cells after trypan blue staining. These experiments showed that while the CD6-ADC killed the proliferating MOLT-4 T cells in a concentration-dependent manner, it had no significant detrimental effect on the proliferating Raji B cells that do not express CD6. These results indicate that the CD6-ADC selectively killed proliferating T cells while sparing non-CD6-expressing cells even they are actively dividing.

### CD6-ADC Eliminates Antigen-Specific Autoreactive T Cells in Vitro

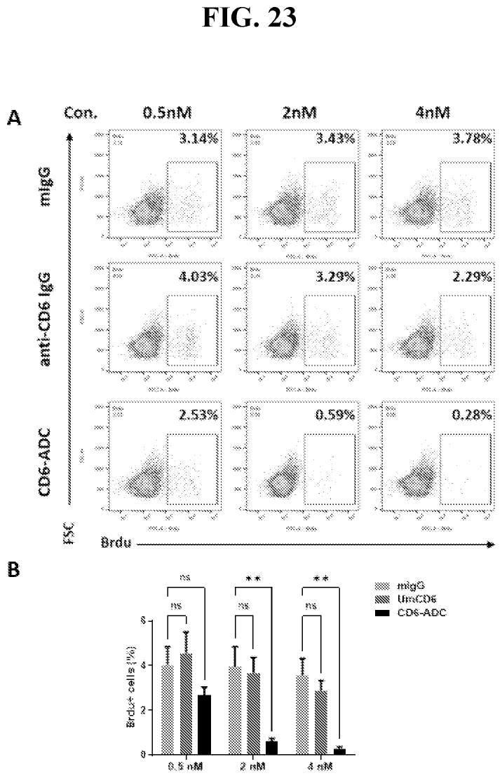

To examine the potential of CD6-ADC in eliminating antigen-specific pathogenic T cells, we immunized CD6 humanized mice with an uveitogenic IRBP peptide, then collected the spleens 12 days later. We set up an antigen-specific recall assay using the splenocytes in the presence of different concentrations of CD6-ADC, the anti-CD6 mAb or the control IgG. To identify the proliferating cells, we also added BrdU in the cultures. In 3 days, we quantitated the percentages of total CD4+ T cells as well as the proliferating BrdU+ CD4+ T cells in each well by flow cytometry. See, FIG. 23. These experiments showed that in the splenocytes analyzed, CD4+ T cells accounted for 30-35% of all cells, and only 4-5% of the CD4+ T cells were IRBP-responsive proliferating cells (BrdU+), which are consistent with the previous reports. In addition, CD6-ADC, but not the anti-CD6 mAb nor the IgG, significantly reduced the numbers of proliferating BrdU+ CD4+ T cells in a concentration-dependent manner in the cultures. These results demonstrated that CD6-ADC selectively killed the IRBP-specific proliferating CD4+ T cells that lead to autoimmune uveitis.

### CD6-Targeted ADC Suppresses the Development of Uveitis Induced by an Adoptive Transfer of Pre-Activated Uveitogenic T Cells

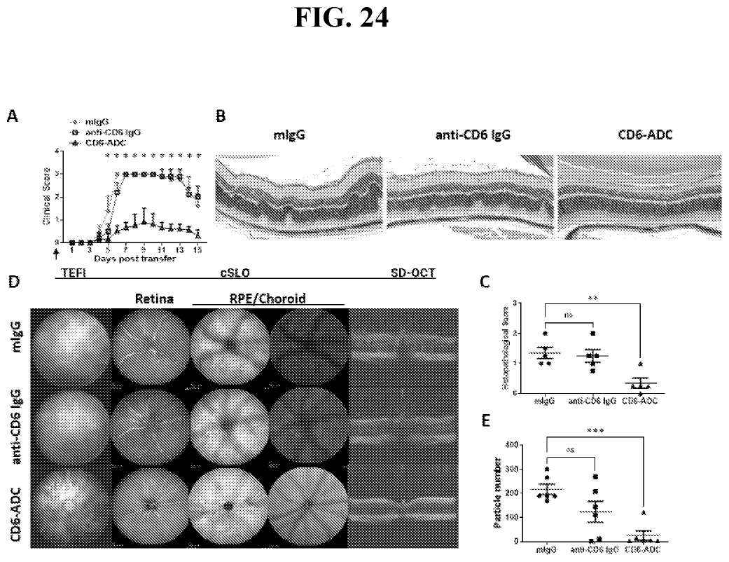

We then tested the treatment efficacy of the CD6-ADC in treating uveitis induced by an adoptive transfer of pre-activated uveitogenic T cells. In brief, following our previously published protocol, we amplified the autoreactive T cells from IRBP-immunized CD6-humanized mice in vitro, then adoptively transferred the pre-activated uveitogenic T cells into naïve mice to induce uveitis. After the adoptive transfer, we randomly divided the mice into 3 groups and treated them with 0.5 mg/kg of the anti-CD6 ADC, anti-CD6 mAb or control IgG. Again, we monitored the development of uveitis daily by indirect ophthalmoscopy and analyzed the mouse retina by OCT and SLO at day 8 together with ocular histopathological analyses. See, FIG. 24. All these studies showed that the dose given, administration of the CD6-ADC, but not the parent anti-CD6 IgG or the control IgG, significantly protected the mice from retinal inflammation induced by the uveitogenic T cells, even though the treatment with the anti-CD6 IgG slightly delayed the disease onset at the dose given.

### CD6-ADC Reverses the Progress of Uveitis Induced by Active Immunization

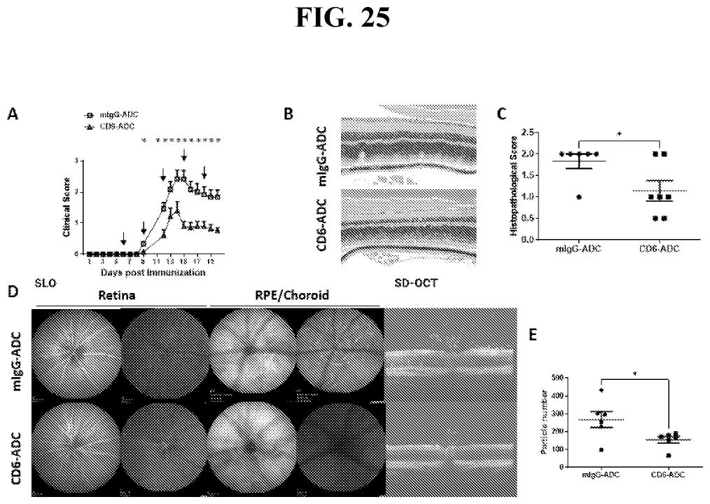

In addition to the above adoptive transfer-induced passive model of EAU, we also tested the treatment efficacy of the CD6-ADC in an autoimmune uveitis model induced by active immunization. In brief, we immunized CD6 humanized mice with the IRBP peptide, and 6 days later, we confirmed that all the mice developed uveitis by SLO as indicated by the presence of hyperfluorescent leukocytes in the retina. We thus randomly divided and treated the mice with either CD6-ADC or a control ADC (0.5 mg/kg), then monitored the progress of uveitis daily by indirect ophthalmoscopy and recorded their clinical scores. In addition, we also analyzed the mouse retina by OCT and SLO on day 14. Finally, at the end of the experiment, we collected eyes for histopathological analyses, and spleens for antigen-specific Th1/Th17 response assays. We found that CD6-ADC, but not the control ADC, significantly attenuated uveitis in the treated mice as analyzed by all the ocular imaging techniques. See, FIG. 25. Besides, autoantigen-specific Th1 and Th17 cells were markedly reduced in the CD6-ADC-treated mice than the control ADC-treated mice.

### CD6-ADC Treats a Pre-Clinical Model of GVHD

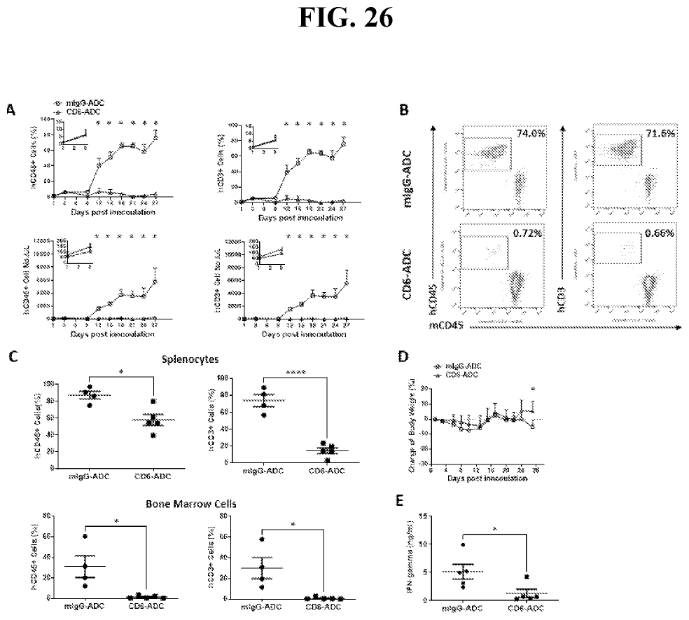

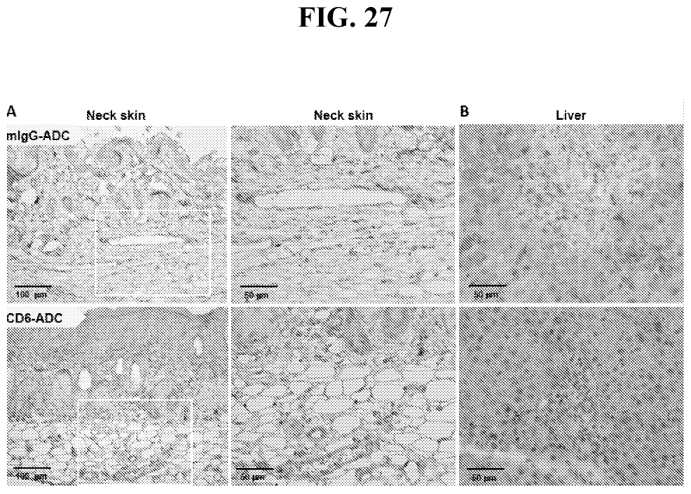

To test the efficacy of the CD6-ADC in treating other T cell-mediated disorders in addition to autoimmune diseases such as autoimmune uveitis, we used a xenogeneic GVHD model. In brief, we infused irradiated NSG mice with fresh human PBMC, then waited 3 days until detecting that the infused human T cells were activated and expanding in the blood by flow cytometry. We therefore treated half of the mice with CD6-ADC (0.5 mg/kg) and the other half with the same dose of control-ADC, and monitored percentages and absolute numbers of circulating human T cells twice a week by flow cytometry to assess the GVHD development until day 27. At the end of the experiments, we also collected different tissues for histopathological analyses. See, FIG. 26. These studies showed that compared with mice treated with the control ADC in which more than 80% of the leukocytes in the blood were human CD45+ CD3+ T cells, mice treated with CD6-ADC only had less than 1% of human CD45+ CD3+ T cells in the blood. In addition to the striking contrast of human T cell percentages and numbers in the peripheral blood, mice treated with CD6-ADC also showed drastically reduced percentages of human T cells in the bone marrow and spleens. Histopathological examinations of different tissues confirmed these hematological analysis results, showing that CD6-ADC treatment markedly reduced human T cell infiltration in multiple organs such as the skin and liver, thus significantly attenuated GVHD. See FIG. 27.

A “holy grail” of treating autoimmune diseases that are mediated by pathogenic T cells is to selectively target these autoreactive T cells while sparing the normal quiescent T cells as well as other tissue cells. The CD6-ADC in this Example appear to achieve this goal because: 1) CD6 is almost exclusively expressed on T cells, the other cells known to express CD6 are B1a cells which account for less than 1% of the total B cells and some natural killer (NK) cells; and 2) MMAE, being an anti-mitotic drug, kills actively proliferating cells. Even though CD6 is present on all T cells, under normal conditions, resting T cells are not actively proliferating therefore these quiescent T cells are not sensitive to the MMAE-mediated killing. On the contrary, the autoreactive pathogenic T cells are actively dividing, which become victims of the CD6-ADC mediated killing.

To the best of our knowledge, so far only one ADC is under active development for treating autoimmune diseases. This ADC is generated by conjugating amanitin, a RNA polymerase II inhibitor onto a mAb against CD45, which is present on all leukocytes including T cells, B cells, NK cells, eosinophils, basophils, monocytes, macrophages and neutrophils. This ADC is highly effective in treating models of MS and GVHD, as well as inflammatory arthritis, and the company website reported that this ADC is currently in IND-enabling studies for clinical evaluations. Although this CD45-targeted ADC demonstrates that applications of ADC are indeed not limited to tumor immunotherapy but can be extended in autoimmune disease treatment, it is significantly different from our CD6-ADC. First, unlike CD45, which is expressed in all leukocytes and some stem cells, CD6 is primarily expressed on T cells. Thus, unlike the nonspecific cytotoxic effects of the CD45-directed ADC targeting all leukocytes, our therapy targets T cells, therefore should not lead to systemic immunosuppression and the related severe side effects. Second, the payload used in the CD45-targeted ADC, amanitin, kills both proliferating and quiescent cells, while the MMAE used in our CD6-ADC is a mitotic toxin thus only killing proliferating cells. By combining the T cell selectivity of the anti-CD6 mAb and the proliferating cell selectivity of the payload MMAE, our CD6-ADC should, in general, have a better safety profile and less side effects by selectively targeting proliferating T cells only. Indeed, all the treated mice in our studies tolerated the CD6-ADC well without any apparent issues.

It has been previously reported that the parental anti-CD6 mAb used in the CD6-ADC development alone is effective in treating mouse models of autoimmune disease such as multiple sclerosis (MS) and rheumatoid arthritis (RA) by suppressing T cell responses without depleting the CD6+ T cells. The CD6-ADC should have significantly greater treatment efficacy than its parental “naked” mAb because of the potent payload conjugated. Indeed, in previous reports, when given at˜4 mg/kg (˜100 μg/mouse), the anti-CD6 mAb was very effective in treating models of MS and RA, but in the treatment experiments described in this Example, we found that at the dose given which was 0.5 mg/kg (˜12 μg/mouse), even though CD6-ADC significantly suppressed the development of uveitis after the adoptive transfer of pre-activated uveitogenic T cells, the same dose of the “naked” anti-CD6 mAb only delayed the development of uveitis and moderately attenuated retina inflammation in the treated mice within the first week of uveitis development. These data indicate that the CD6-ADC has a significantly heightened treatment efficacy than the parent anti-CD6 mAb in treating autoimmune diseases with much lower doses required for effectiveness, which could possibly lead to many benefits including reduced costs and decreased potential side effects.

When uveitis patients come to the clinic, they already have developed uveitogenic T cells and/or shown signs of uveitis. We started the treatment studies after adoptive transfer of pre-activated uveitogenic T cells in the passive model or after the mice showed signs of uveitis in the active model, both of which faithfully mimic the patient situations in the clinic. All the ocular imaging techniques (SLO, OCT and indirect ophthalmoscopy) that we used to examine the mouse retina are also commonly used for uveitis diagnosis and evaluations in the clinic. Thus the positive treatment data from these pre-clinical models of autoimmune uveitis provides a strong rationale for CD6-ADC as a drug for human patients.

In addition to many autoimmune diseases such as autoimmune uveitis, GVHD is another disorder mediated by pathogenic T cells. GVHD occurs in most patients after allogeneic bone marrow (BM) transplantation, which is the last resort for diseases such as sickle cell anemia, paroxysmal nocturnal hemoglobinuria and many hematologic malignancies. Despite the understanding that activated and expanded donor T cells damage the host tissues to cause GVHD, currently available therapeutic options are limited, unsatisfactory and with severe side effects. We employed a xeno-GVHD model, which is commonly used to evaluate potential drug candidates for treating GVHD in humans. In this pre-clinical model of GVHD, we found that even given at a low dose of 0.5 mg/kg after the pathogenic T cells are activated and expanding in vivo, the CD6-ADC, but not the control-ADC, efficiently killed the pathogenic expanding human T cells, leading to significantly reduced numbers of human T cells in vivo and consequently, markedly attenuated or even diminished pathology in multiple organs such as livers, spleens and skins. These data suggest that the CD6-ADC could be a therapeutic option for GVHD in addition to autoimmune diseases like autoimmune uveitis.

When patients are infected, pathogen-specific T cells are activated and start to proliferate. If these patients are still under the treatment of the CD6-ADC, their pathogen-specific T cells will also be sensitive to the CD6-ADC-mediated killing, which could lead to opportunistic infections. To mitigate these complications, in which cases, the CD6-ADC treatment regimen can be halted until antibiotics and/or anti-viral drugs are administrated to help the patients control the invading pathogens.

In summary, the CD6-ADC that we developed selectively kills proliferating pathogenic T cells and is highly effective in reversing disease progression in two pre-clinical models of autoimmune uveitis as well as a pre-clinical model of GVHD even when given at a low dose. These results suggest that the CD6-ADC is a drug for treating pathogenic T cells-mediated disorders, including but not limited to diseases like autoimmune uveitis, multiple sclerosis, rheumatoid arthritis, GVHD, and transplantation rejections.

### Example 2

### CD6-Targeted Antibody-Drug Conjugate as Therapy for B1-Cell Mediated Disorders

Mantle cell lymphoma (MCL) is an aggressive B 1-cell non-Hodgkin lymphoma with poor clinical prognosis and no cure (1). These tumor cells metastasize and invade lymph nodes, spleen, blood, bone marrow, and other tissues and usually kill the patients within 2-3 years of diagnosis (2). Current frontline treatments include the combinations of cytotoxic chemotherapeutic agents or strenuous chemo-immunotherapy with subsequent stem cell transplantation (3,4). Despite all the severe side effects from these available management options, while MCL patients tend to respond to these treatments initially, most of the patients relapse later or become refractory (5,6). Thus, it is of great clinical importance and urgency to develop new drugs targeting these malignant B cell tumors.

The first and one of the most important steps in developing a targeted therapy is the identification of a target molecule on the MCL cells. It was discovered that all the patient MCL specimen examined express CD6 at high levels, suggesting that CD6 could be a novel therapeutic target for MCL. In studies, we developed a CD6-targeted antibody drug conjugate (ADC) by linking an inactivated form of Monomethyl auristatin E (MMAE), a mitotic toxin and clinically proven payload (7,8) to our high-affinity monoclonal antibody (mAb) against CD6 (see FIG. 5). This ADC is designed to deliver the MMAE into the CD6+ MCL tumor cells. Significantly, being a mitotic toxin, the conjugated MMAE will only kill actively proliferating cells. By combining the selectivity of the anti-CD6 mAb to CD6+ cells, and the selectivity of the mitotic toxin MMAE to proliferating cells, this novel ADC is designed to kill only proliferating CD6+ malignant tumor cells while sparing normal quiescent CD6+ cells and other proliferating but non-CD6 expressing cells.

CD6 is primarily expressed on T cells and a small group of B cells termed B1 cells. CD6, a protein containing 3 extracellular scavenger receptor cysteine-rich (SRCR) domains, was discovered more than 30 years ago as a marker of T cells (19). Later studies also suggested that CD6 is present on a small group of B cells called B1 cells (20). It has been suggested that CD6 is a target for treating T cell-mediated autoimmune diseases, including multiple sclerosis (MS), rheumatoid arthritis, and Sjögren's syndrome (22). Interest in this field increased significantly when several groups discovered that CD6 is a risk gene for MS (23-25). Recently, itolizumab, an anti-CD6 mAb developed in Cuba, has been approved for treating psoriasis and COVID-19 in India (26,27). During the last 10 years, by developing and studying CD6 knockout (KO) mice, we have found that the lack of CD6 activity protected mice in several T cell-mediated autoimmune disease models, including models of autoimmune uveitis (11), MS(9) and RA(13). We also confirmed using CD6 KO mice that CD6 is indeed present on B1 cells but not any other B cells or myeloid cells (14).

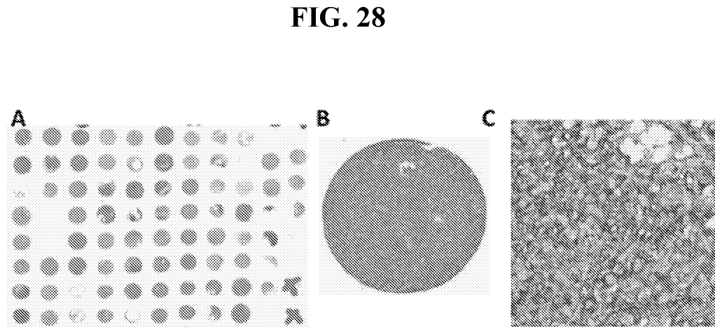

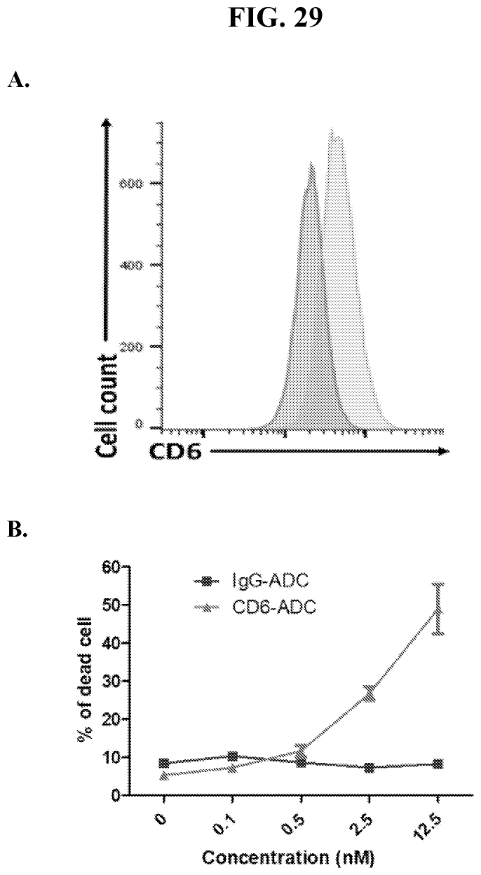

With the data showing that all MCL patient samples that we have examined express CD6 at high levels (FIG. 28), since normal T cells are not proliferating in patients even though they are CD6+ and the MCL cells are actively dividing, we could take advantage of our identified anti-CD6 mAb to develop an ADC to selectively kill the MCL cells as a new therapeutic approach for MCL patients. We thus generated the ADC by conjugating an inactivated form of the mitotic toxin MMAE onto our identified anti-CD6 mAb via the same cleavable VC-PAB linker (FIG. 5). This ADC, by design, should selectively kill proliferating MCL cells while sparing the normal T cells and other tissue cells. We further showed that this novel ADC, but not the parent “naked” anti-CD6 mAb nor the control IgG efficiently killed actively proliferating MCL cells in vitro (FIG. 29), demonstrating its potential as a new drug for MCL.

All MCL patient specimens examined are CD6+. An MCL patient tissue microarray that contains˜200 tumor samples was employed. The array was stained with our anti-CD6 mAb and the stained slides were examined. All the samples, except a few that lacked the tumor tissues, are strongly stained for CD6 (FIG. 28). These results not only demonstrate for the first time that MCL cells express CD6 on the surface at high levels, but also suggest that CD6 is a novel therapeutic target for patients with MCL, especially the patients who are refractory to the currently available treatments.

We developed a CD6-targeted ADC by conjugating an inactive form of MMAE onto our identified anti-CD6 mAb using a kit developed by CellMosaic Inc (Boston, MA) (FIG. 5A). The target payload to antibody ratio is estimated to be 4:1 according to the spectroscopy analysis measuring OD418/OD280. To test the potential of this novel ADC in killing MCL cells, we first determined that SP53, a well-established human MCL cell line (30,31) is CD6+. We then incubated the SP53 MCL cells with different concentrations of the CD6-ADC, or the control ADC (non-specific mouse IgGs conjugated with the MMAE using the same kit), and assessed MCL cell death in 72 hrs by trypan blue staining. We found that the CD6-targeted ADC, but not the anti-CD6 mAb nor the control IgG, potently killed the MCL cells with an in these in vitro assays (FIG. 29).

### REFERENCES

1. Cortelazzo et al., Crit Rev Oncol Hematol. 2020;153:103038.
2. Martin et al., Journal of Clinical Oncology. 2009;27(8):1209-1213.
3. Hanel and Epperla, Journal of Hematology & Oncology. 2020;13(1):79.
4. Cheminant et al., Ann Lymphoma 2020;4:2.
5. Schieber et al., F1000Res. 2018;7.
6. Kumar et al., Blood Cancer Journal. 2019;9(6):50.
7. Richardson et al., Oncologist. 2019;24(5):e180-e187.
8. Malecek et al., Expert Opin Biol Ther. 2020:1-9.
9. Li et al., Proc Natl Acad Sci USA. 2017;114(10):2687-2692.
10. Enyindah-Asonye et al., Proc Natl Acad Sci USA. 2017;114(33):E6912-E6921.
11. Zhang et al., J Autoimmun. 2018;90:84-93.
12. Consuegra-Fernandez et al., Autoimmun Rev. 2018;17(5):493-503.
13. Li et al., Arthritis Rheumatol. 2020.
14. Enyindah-Asonye et al., J Biol Chem. 2017;292(2):661-671.
15. Han et al., BMC Res Notes. 2018;11(1):229.
16. Singh et al., Curr Clin Pharmacol. 2018;13(2):85-99.
17. Khongorzul et al., Mol Cancer Res. 2020;18(1):3-19.
18. Shea et al., Curr Hematol Malig Rep. 2020;15(1):9-19.
19. Kamoun et al., J Immunol. 1981;127(3):987-991.
20. Alonso et al., Journal of Autoimmunity. 2010;35(4):336-341.
21. Braun et al., J Innate Immun. 2011;3(4):420-434.
22. Ramos-Casals et al., Rheumatology (Oxford). 2001;40(9):1056-1059.
23. Swaminathan et al., PLoS One. 2013;8(4):e62376.
24. De Jager et al., Nat Genet. 2009;41(7):776-782.
25. International Multiple Sclerosis Genetics C. PLoS One. 2011;6(4):e18813.
26. Jayaraman et al., Nat Biotechnol. 2013;31(12):1062-1063.
27. Menon et al., Clin Cosmet Investig Dermatol. 2015;8:215-222.
28. Alonso et al., Hybridoma (Larchmt). 2008;27(4):291-301.
29. Starkebaum et al., Int J Cancer. 1991;49(2):246-253.
30. Daibata et al., J Virol. 1996;70(12):9003-9007.
31. Amin et al., Arch Pathol Lab Med. 2003;127(4):424-431.
32. Pham et al., Clin Cancer Res. 2018;24(16):3967-3980.
33. Zhang et al, Cancer Res. 2016;76(21):6410-6423.
34. Wang et al., Leukemia. 2008;22(1):179-185.
35. Zhang et al., Blood. 2017;130(6):763-776.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of treating a subject comprising:
administering antibody drug conjugate (ADC) to a subject with a disorder, wherein said disorder is a T-cell mediated disorder, a B1-cell mediated disorder, a T-cell lymphoma, or a B-cell lymphoma, and
wherein said ADC comprises:
a) an anti-CD6 antibody, or CD6 binding portion thereof, and
b) a mitotic inhibitor drug.

2. The method of claim 1, wherein said mitotic inhibitor drug comprises monomethyl auristatin E (MMAE).

3. The method of claim 1, wherein said mitotic inhibitor drug is selected from the group consisting of: vincristine, eribulin, paclitaxel, paclitaxel protein-bound, docetaxel, estramustine, etoposide, ixabepilone, cabazitaxel, vincristine liposome, vinorelbine, vincristine, paclitaxel, etoposide, vinblastine, etoposide, and teniposide.

4. The method of claim 1, wherein said subject has said T-cell mediated disorder, and wherein said T-cell mediated disorder comprises autoimmune uveitis.

5. The method of claim 1, wherein said subject has said T-Cell mediated disorder, and wherein said T-cell mediated disorder is selected from the group consisting of: rheumatoid arthritis (RA), type 1 diabetes, Multiple sclerosis, graft vs host disease, Celiac disease, and Sjögren' s syndrome.

6. The method of claim 1, wherein said ADC further comprises a cleavable linker.

7. The method of claim 1, wherein said anti-CD6 antibody, or CD6 binding portion thereof, comprises one or more CDRs from Table 1.

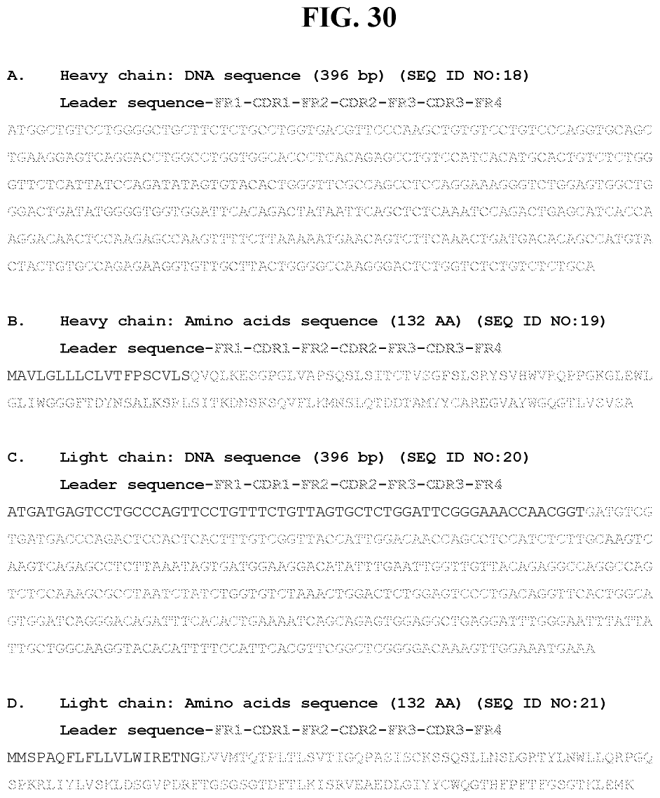

8. The method of claim 1, wherein said anti-CD6 antibody, or CD6 binding portion thereof, comprises one or more variable regions shown in FIGS. 18-21 and 30.

9. The method of claim 1, wherein said subject is a human.

10. The method of claim 1, wherein said ADC is administered to said subject at a dosage of about 0.1-2 mg/kg.

11. The method of claim 1, wherein said subject has said B-cell lymphoma

12. The method of claim 11, wherein said B-cell lymphoma is Mantle cell lymphoma.

13. The method of claim 1, wherein said subject has said T-cell lymphoma.

14. A composition comprising: an antibody drug conjugate (ADC) comprising:
a) an anti-CD6 antibody, or CD6 binding portion thereof, and
b) a mitotic inhibitor drug.

15. The composition of claim 14, wherein said mitotic inhibitor drug comprises monomethyl auristatin E (MMAE).

16. The composition of claim 14, wherein said mitotic inhibitor drug is selected from the group consisting of: vincristine, eribulin, paclitaxel, paclitaxel protein-bound, docetaxel, estramustine, etoposide, ixabepilone, cabazitaxel, vincristine liposome, vinorelbine, vincristine, paclitaxel, etoposide, vinblastine, etoposide, and teniposide.

17. The composition of claim 14, wherein said ADC further comprises a cleavable linker.

18. The composition of claim 14, wherein said anti-CD6 antibody, or CD6 binding portion thereof, comprises one or more CDRs from Table 1.

19. The method of claim 14, wherein said anti-CD6 antibody, or CD6 binding portion thereof, comprises one or more variable regions shown in FIGS. 18-21 and 30.

20. An in vitro system comprising:
a) an antibody drug conjugate (ADC) comprising: i) an anti-CD6 antibody, or CD6 binding portion thereof, and ii) a mitotic inhibitor drug; and
b) a T-cell lymphoma cell, or a B-cell lymphoma cell.


SEQUENCE LISTING (21 sequences)

SEQ ID NO: 1 - protein, 113 aa, Artificial sequence
  Synthetic
QVQLQESGPGLVKPSETLSLTCTVSGGSISRYSVHWIRQPPGKGLEWIGLIWGGGFTDYNSALKSRVTISVDTSKNQFSLKLSSVTAADTAVYYCAREGVAYWGQGTLVTVSS

SEQ ID NO: 2 - protein, 113 aa, Artificial sequence; identical to SEQ ID NO: 1
  Synthetic

SEQ ID NO: 3 - protein, 111 aa, Artificial sequence
  Synthetic
DVVMTQSPLSLPVTLGQPASISCKSSQSLLNSDGRTYLNWFQQRPGQSPRRLIYLVSKLDSGVPDRFSGSGGTDFTLKISRVEAEDVGVYYCWQGTHFPFTFGPGTKVDIK

SEQ ID NO: 4 - protein, 113 aa, Artificial sequence
  Synthetic
QVQLQESGPGLVKPSETLSLTCTVSGGSISRYSVHWIRQPPGKGLEWIGLIWGGGFTDYNSALKSRVSITVDTSKNQFSLKLSSVTAADTAVYYCAREGVAYWGQGTLVTVSS

SEQ ID NO: 5 - protein, 111 aa, Artificial sequence
  Synthetic
DVVMTQSPLSLPVTLGQPASISCKSSQSLLNSDGRTYLNWFQQRPGQSPKRLIYLVSKLDSGVPDRFSGSGGTDFTLKISRVEAEDVGVYYCWQGTHFPFTFGPGTKVDIK

SEQ ID NO: 6 - protein, 113 aa, Artificial sequence
  Synthetic
QVQLQESGPGLVKPSETLSLTCTVSGFSLSRYSVHWVRQPPGKGLEWLGLIWGGGFTDYNSALKSRLTISKDNSKNQVSLKLSSVTAADTAVYYCAREGVAYWGQGTLVTVSS

SEQ ID NO: 7 - protein, 111 aa, Artificial sequence; identical to SEQ ID NO: 3
  Synthetic

SEQ ID NO: 8 - protein, 224 aa, Artificial sequence
  Synthetic
QVQLQESGPGLVKPSETLSLTCTVSGFSISRYSVHWIRQPPGKGLEWIGLIWGGGFTDYNSALKSRVTISKDNSKNQVSLKLSSVTAADTAVYYCAREGVAYWGQGTLVTVSSDVVMTQSPLSLPVTLGQPASISCKSSQSLLNSDGRTYLNWFQQRPGQSPRRLIYLVSKLDSGVPDRFSGSGGTDFTLKISRVEAEDVGVYYCWQGTHFPFTFGPGTKVDIK

SEQ ID NO: 9 - protein, 113 aa, Artificial sequence
  Synthetic
QVQLQESGPGLVKPSETLSLTCTVSGGSLSRYSVHWVRQPPGKGLEWLGLIWGGGFTDYNSALKSRLTISVDTSKNQFSLKLSSVTAADTAVYYCAREGVAYWGQGTLVTVSS

SEQ ID NO: 10 - protein, 111 aa, Artificial sequence; identical to SEQ ID NO: 3
  Synthetic

SEQ ID NO: 11 - protein, 113 aa, Artificial sequence
  Synthetic
QVQLQESGPGLVKPSETLSLTCTVSGFSLSRYSVHWIRQPPGKGLEWIGLIWGGGFTDYNSALKSRVTISKDNSKNQVSLKLSSVTAADTAVYYCAREGVAYWGQGTLVTVSS

SEQ ID NO: 12 - protein, 111 aa, Artificial sequence; identical to SEQ ID NO: 3
  Synthetic

SEQ ID NO: 13 - protein, 113 aa, Artificial sequence
  Synthetic
QVQLQESGPGLVKPSETLSLTCTVSGGSISRYSVHWVRQPPGKGLEWLGLIWGGGFTDYNSALKSRLTISVDTSKNQFSLKLSSVTAADTAVYYCAREGVAYWGQGTLVTVSS

SEQ ID NO: 14 - protein, 111 aa, Artificial sequence; identical to SEQ ID NO: 3
  Synthetic

SEQ ID NO: 15 - protein, 113 aa, Artificial sequence
  Synthetic
QVQLQESGPGLVKPSETLSLTCTVSGGSLSRYSVHWVRQPPGKGLEWIGLIWGGGFTDYNSALKSRLTISVDTSKNQVSLKLSSVTAADTAVYYCAREGVAYWGQGTLVTVSS

SEQ ID NO: 16 - protein, 113 aa, Artificial sequence; identical to SEQ ID NO: 1
  Synthetic

SEQ ID NO: 17 - protein, 20 aa, Artificial sequence
  Synthetic
SGIPYIISYLHPGNTILHVD

SEQ ID NO: 18 - DNA, 396 bp, Artificial sequence
  Synthetic
atggctgtcctggggctgcttctctgcctggtgacgttcccaagctgtgtcctgtcccaggtgcagctgaaggagtcaggacctggcctggtggcaccctcacagagcctgtccatcacatgcactgtctctgggttctcattatccagatatagtgtacactgggttcgccagcctccaggaaagggtctggagtggctgggactgatatggggtggtggattcacagactataattcagctctcaaatccagactgagcatcaccaaggacaactccaagagccaagttttcttaaaaatgaacagtcttcaaactgatgacacagccatgtactactgtgccagagaaggtgttgcttactggggccaagggactctggtctctgtctctgca

SEQ ID NO: 19 - protein, 132 aa, Artificial sequence
  Synthetic
MAVLGLLLCLVTFPSCVLSQVQLKESGPGLVAPSQSLSITCTVSGFSLSRYSVHWVRQPPGKGLEWLGLIWGGGFTDYNSALKSRLSITKDNSKSQVFLKMNSLQTDDTAMYYCAREGVAYWGQGTLVSVSA

SEQ ID NO: 20 - DNA, 396 bp, Artificial sequence
  Synthetic
atgatgagtcctgcccagttcctgtttctgttagtgctctggattcgggaaaccaacggtgatgtcgtgatgacccagactccactcactttgtcggttaccattggacaaccagcctccatctcttgcaagtcaagtcagagcctcttaaatagtgatggaaggacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggaatttattattgctggcaaggtacacattttccattcacgttcggctcggggacaaagttggaaatgaaa

SEQ ID NO: 21 - protein, 132 aa, Artificial sequence
  Synthetic
MMSPAQFLFLLVLWIRETNGDVVMTQTPLTLSVTIGQPASISCKSSQSLLNSDGRTYLNWLLQRPGQSPKRLIYLVSKLDSGVPDRFTGSGSGTDFTLKISRVEAEDLGIYYCWQGTHFPFTFGSGTKLEMK